(19) United States
(12) Patent Application Publication
(10) Pub. No.: US 2019/0282812 A1
(43) Pub. Date: Sep. 19, 2019

(54) METHOD FOR NON-INVASIVE ENHANCEMENT OF DEEP SLEEP

(71) Applicant: Teledyne Scientific & Imaging, LLC, Thousand Oaks, CA (US)

(72) Inventors: Stephen Simons, Raleigh, NC (US); Patrick Connolly, Cary, NC (US); Renee Shimizu, Raleigh, NC (US); Mario Aguilar-Simon, Chapel Hill, NC (US)

(21) Appl. No.: 16/354,029

(22) Filed: Mar. 14, 2019

Related U.S. Application Data
(60) Provisional application No. 62/642,871, filed on Mar. 14, 2018.

(51) Int. Cl. A61N 1/36; A61B 5/0478; A61B 5/00; A61B 5/0496; A61B 5/0408; A61N 1/20; A61M 21/02

(52) U.S. Cl. CPC A61N 1/36031; A61B 5/0478; A61B 5/4812; A61N 1/36025; A61M 2021/0072; A61B 5/0496; A61B 5/04085; A61N 1/20; A61M 21/02; A61B 5/6803

(57) ABSTRACT

Provided is an apparatus, system, and method for targeted sleep enhancement. A computer processing circuit receives a plurality of EEG signals from a plurality of spatially separated EEG sensors configured to be located on the head of a subject. The computer processing circuit executes machine executable instructions to: receive and process the plurality of EEG signals; determine that the subject is in sleep stage 3 based on a specific EEG signal of the processed plurality of EEG signals; determine a period of at least one of quiescent and asynchronous brain activity of the subject, wherein the period is determined based on the processed plurality of EEG signals; and deliver a transcranial electrical stimulation through the plurality of stimulation electrodes during the period of quiescent brain activity.

### PRIORITY CLAIM

The present application claims priority under 35 U.S.C. § 119(e) to U.S. Provisional Patent Application No. 62/642,871, titled METHOD FOR NON-INVASIVE ENHANCEMENT OF DEEP SLEEP, filed on Mar. 14, 2018, the disclosure of which is herein incorporated by reference in its entirety.

### STATEMENT OF INCORPORATION BY REFERENCE

U.S. application Ser. No. 15/720,621, filed Sep. 29, 2017, and U.S. Provisional Application No. 62/403,318, filed Oct. 3, 2016, are herein incorporated by reference in their entirety by this application.

### STATEMENT OF GOVERNMENTAL INTEREST

The subject matter described in the present disclosure was developed with U.S. Government support under DARPA RAM Replay program contract number W911NF-16-2-0007. The U.S. Government has certain rights in the subject matter.

### FIELD OF TECHNOLOGY

The present disclosure is directed generally to an apparatus, system, and method for targeted sleep enhancement for humans in a closed-loop system.

### BACKGROUND

Sleep can be classified according to various stages, including wake, rapid eye movement (REM), non-rapid eye movement (NREM) stage 1, NREM stage 2, NREM stage 3. Some sleep classifications include a NREM stage 4 as well. Slow wave sleep (SWS) is a deep sleep that occurs during NREM stage 3 (and NREM stage 4 in such sleep stage classifications). SWS is characterized by a larger frequency of slow waves (slow oscillations), which are delta brain waves that can be detected via electroencephalography (EEG). During SWS, the brain becomes less responsive to external stimuli. Accordingly, SWS can be referred to as deep sleep, which is the most difficult stage of sleep for a person to awaken from and is typically thought to represent one of the more recuperative aspects of sleep. Applying electrical stimulation to the brain of the person can result in increased deep sleep.

### SUMMARY

In one aspect, the present disclosure is directed to a system for targeted sleep enhancement. The system comprises a plurality of spatially separated electroencephalography (EEG) sensors configured to be located on the head of a subject to generate a plurality of EEG signals; a plurality of stimulation electrodes configured to be located on the head of the subject; and a computer processing circuit configured to receive and process the plurality of EEG signals. The computer processing circuit is also programmed to: determine that the subject is in a sleep stage 3 based on a specific EEG signal of the processed plurality of EEG signals; determine a period of at least one of quiescent and asynchronous brain activity of the subject, wherein the period is determined based on the processed plurality of EEG signals; and deliver a transcranial electrical stimulation through the plurality of stimulation electrodes during the period of quiescent brain activity.

In another aspect, the present disclosure is directed to a headband configured to be used in conjunction with a computer processing circuit for targeted sleep enhancement and configured to be worn on the head of an individual. The headband comprises a plurality of spatially separated electroencephalography (EEG) sensors configured to be located on the head of the subject to generate a plurality of EEG signals and a plurality of stimulation electrodes configured to be located on the head of the subject. The computer processing circuit is programmed to: receive and process the plurality of EEG signals; determine that the subject is in a sleep stage 3 based on a specific EEG signal of the plurality of EEG signals; determine that the subject is in one of a non rapid-eye movement (NREM) sleep stage 2 or NREM sleep stage 3 based on a specific EEG signal of the processed plurality of EEG signals; and deliver a series of pulses of transcranial electrical stimulation through the plurality of stimulation electrodes during a period of quiescent brain activity of the subject.

In another aspect, the present disclosure is directed to a method for targeted sleep enhancement using an electroencephalography (EEG) headband comprising a computer processing circuit coupled to a memory storing machine executable instructions, a plurality of spatially separated EEG sensors configured to be located on the head of the subject to generate a plurality of EEG signals, and a plurality of stimulation electrodes configured to be located on the head of the subject. The method comprising executing, by the computer processing circuit, the machine executable instructions to perform targeted deep sleep enhancement, wherein performing targeted deep sleep enhancement comprises: determining that the subject is in a sleep stage 3 based on a specific EEG signal of the plurality of EEG signals; determining that there is an ongoing slow oscillation based on detection of a cortical down state to up state transition event; and delivering the transcranial electrical stimulation.

The foregoing summary is illustrative only and is not intended to be in any way limiting. In addition to the illustrative aspects and features described above, further aspects and features will become apparent by reference to the drawings and the following detailed description.

### FIGURES

The patent or application file contains at least one drawing executed in color. Copies of this patent or patent application publication with color drawing(s) will be provided by the Office upon request and payment of the necessary fee.

The novel features described herein are set forth with particularity in the appended claims. Various aspects, however, both as to organization and methods of operation may be better understood by reference to the following description, taken in conjunction with the accompanying drawings as follows:

FIG. 1 is a diagram that represents the closed-loop operation of a targeted deep sleep enhancement system, according to one aspect of the present disclosure.

FIGS. 2-6 illustrate various views of hardware components of a targeted deep sleep enhancement system for monitoring brain activity and stimulating ongoing slow oscillations in the brain during NREM stage 2 or stage 3 sleep, according to various aspects of the present disclosure.

FIG. 7 is a diagram of a plurality of EEG signals that are sensed by a plurality of EEG sensors, according to one aspect of the present disclosure.

FIG. 8 depicts a hypnogram for determining the stages of sleep of a subject, according to one aspect of the present disclosure.

FIG. 9 graphically depicts a cortical down-state to up-state transition event in a digital EEG waveform, according to one aspect of the present disclosure.

FIG. 10 is a flow diagram of a sleep stage detection algorithm, according to one aspect of the present disclosure.

FIG. 11 is a flow diagram of a sleep stage detection algorithm that does not check for REM sleep stage, according to one aspect of the present disclosure.

FIG. 12 illustrates an architectural or component view of a computing system that may be employed with the closed-loop deep sleep enhancement methodology described in connection with FIGS. 1-11, according to various aspects of the present disclosure.

FIG. 13 is a graph illustrating a duration spent in each sleep stage, according to one aspect of the present disclosure.

FIG. 14 is a graph plotting potential difference relative to time and illustrates a period of time comprising a stimulation cycle, according to one aspect of the present disclosure.

FIGS. 15A-15C are various graphs illustrating differences in memory performance for two groups of subjects at a post-nap and a delayed memory recall test in which the first group of subjects received a transcranial electrical stimulation while the second group of subjects received a sham stimulation, according to various aspects of the present disclosure.

FIG. 16 is a graph illustrating a comparison between a duration spent in each sleep stage for subjects receiving a transcranial electrical stimulation and subjects receiving a sham stimulation, according to one aspects of the present disclosure.

FIGS. 17A-17B are graphs of power spectral density and topographic maps of an EEG field in a two dimensional circular view for subjects in sham, electrical stimulation, and the difference between sham and electrical stimulation conditions, according to various aspects of the present disclosure.

FIG. 18 includes topographic maps of an EEG field in a two dimensional circular view for subjects sham, electrical stimulation, and the difference between sham and electrical stimulation conditions, in which the maps are categorized by slow oscillation rate, up-state duration, up-state amplitude, fast spindle energy, and slow spindle energy, according to one aspect of the present disclosure.

FIGS. 19A-19H are various three dimensional models illustrating current distribution on the scalp and gray matter for bilateral and frontal-mastoid electrode locations, according to various aspects of the present disclosure.

### DESCRIPTION

In the following detailed description, reference is made to the accompanying drawings, which form a part hereof. In the drawings, similar symbols and reference characters typically identify similar components throughout the several views, unless context dictates otherwise. The illustrative aspects described in the detailed description, drawings, and claims are not meant to be limiting. Other aspects may be utilized, and other changes may be made, without departing from the scope of the subject matter presented here.

Before explaining various aspects of surgical devices and generators in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

Also, in the following description, it is to be understood that terms such as front, back, inside, outside, top, bottom and the like are words of convenience and are not to be construed as limiting terms. Terminology used herein is not meant to be limiting insofar as devices described herein, or portions thereof, may be attached or utilized in other orientations. The various aspects will be described in more detail with reference to the drawings.

One conventional classification of sleep includes four sleep stages: rapid eye movement (REM), non-rapid eye movement (NREM) stage 1, NREM stage 2, NREM stage 3. Alternatively, sleep can be classified such that an additional NREM stage 4 is part of the sleep classification. The brain activity of a person may be monitored via electroencephalography (EEG) to detect sleep stages. During one sleep cycle (the amount of time necessary for the person to transition through the five stages of sleep), various biomarkers or signals can be detected. These biomarkers can be used to apply intervention signals (e.g. electrical stimulation) to the person in a closed-loop manner for enhancing sleep. That is, the timing of the intervention signals is targeted synchronously with low spectral power and/or cross-channel coherence (indicating quiescent and/or asynchronous neural activity) or with naturally occurring neural oscillations such as slow waves. Slow waves (slow oscillations) may characterize slow wave sleep (SWS), which is a stage of deep sleep that occurs during NREM stage 3 (and NREM stage 4 in such classifications). In other words, such slow oscillations dominate NREM stage 3 sleep. The deeper sleep of NREM stage 3 is associated with the restorative properties of sleep as well as memory consolidation.

In one aspect, the present disclosure provides EEG based systems, apparatuses and methods for non-invasive enhancement of deep sleep by enhancing slow oscillations using a closed-loop approach. This sleep enhancement closed-loop approach may comprise gating an intervention signal based on low spectral power and cross-channel coherence or other transition events. To this end, when the person is detected as entering sleep stage 2, sleep stage 3, a weak electrical stimulation such as a 2 milliampere (mA) current can be gated. That is, the interventional signal may be a transcranial electrical stimulation (tES) applied to the person during NREM stage 2 or NREM stage 3. Gating refers to triggering the tES based on various trigger signals in the disclosed closed-loop system. Trigger signals include the detection of periods of asynchronous and/or quiescent brain activity, a large amplitude down-state to up-state transition event (DUPT), and other suitable trigger signals. The trigger signals can be determined based on measured EEG signals of the subject. When tES is applied based on an asynchronous and/or quiescent brain activity trigger signal in a closed-loop methodology, such application of tES may result in a significant increase in the rate of occurrence of subsequent slow oscillations as well as the duration of slow oscillations. As a result, such a triggered application of tES can increase the relative amount of time the person/subject spends in NREM stage 3 sleep versus in NREM stage 1. This corresponds to a greater duration of deep sleep (NREM stage 3) for the subject, thereby achieving a desired enhancement in deep sleep and the associated advantages of greater deep sleep (e.g., greater sleep quality, restoration, and subsequent cognitive performance). This asynchronous and/or quiescent brain activity trigger signal methodology has been experimentally tested, as described in further detail in below.

A DUPT refers to a transition from an unexcited cortical down-state to an excited cortical up-state, which reflects a change from negative to positive mean voltage potential. Large amplitude DUPTs may be characterized as those transitions that exceed a predetermined threshold, such as −50 microvolts (μV) or −80 μV. In the present disclosure, these large amplitude DUPTs may generally be referred to as DUPTs (which indicates an ongoing slow oscillation or K-complex). Detecting DUPTs may comprise identifying a large amplitude slow oscillation or K-complex and tracking or predicting when the mean voltage potential transitions from negative to positive. In aspects in which the DUPT is used to trigger delivery of electrical stimulation, a brief delay or latency may exist between DUPT detection and applying the tES. The tES may be applied in the form of a pulsed transcranial direct current stimulation (tDCS), which could comprise a short series of tDCS pulses for a short duration (e.g., 1 Hertz (Hz)). In particular, following detection of a specific biomarker (e.g., slow oscillation) during sleep, a short burst of tES pulses lasting a total duration of between five to ten seconds may be applied. tES also includes transcranial alternating current stimulation (tACS).

Delivering tDCS stimulation for a short duration (dosage) may be advantageous by allowing the subject's brain to respond to the stimulus (before further stimulation is applied, for example). This short duration can be important because the amount of tDCS applied to the person within a predetermined amount of time can be limited. One standard maximum threshold is that tDCS should not be applied for more than 30 minutes every 7.5 hours, approximately. Accordingly, a longer duration or dosage of tDCS (continuous electrical stimulation for 5 minutes, for example) may be less effective. Specifically, it may be difficult to properly deliver the electrical stimulation with such a longer dosage because EEG stimulation artifacts may prevent identification of the sleep stage that the person is in. For example, with a longer dosage, the person might wake up or enter a different stage of sleep other than sleep stage 2 through 4. In this case, the longer dosage stimulus may not be as effective because the person is not in the desired sleep stage. More generally, the shorter duration of stimulus may beneficially give the brain a chance to respond and a chance for the tDCS delivery to be modified in response. Accordingly, the effects of deep sleep enhancement may be more effective and last longer.

Additionally, a longer dosage of electrical stimulation may cause greater skin irritation compared to a shorter dosage. Applying a short dosage of tDCS stimulation during an ongoing oscillation and allowing the person's brain a chance to respond also enables a reduction in the total amount of stimulation that the person is subjected to. For example, the total dosage of electrical stimulation may generally span a duration of less than 5 minutes, or even significantly less than 5 minutes (as opposed to 30 minutes, for example). Accordingly, by applying short duration tDCS, a longer duration of stimulation throughout the night may be enabled since the standard dosage of 30 minutes per 7.5 hours will not be as quickly exceeded. By applying electrical stimulation in a closed-loop fashion, the present disclosure may provide an increase in the duration and intensity of deep, restorative sleep to increase the sleep quality and subsequent cognitive performance of the person. Moreover, the present disclosure may provide improved sleep quality during restricted sleep and result in the person requiring shorter amounts/time of recovery sleep following sleep deprivation.

In contrast to open-loop approaches, the closed-loop approach of the present disclosure may comprise delivering targeted electrical stimulations. The targeted tES may be delivered during periods of asynchronous and/or quiescent brain activity or during detected ongoing slow oscillations (delta frequency band activity in the person's brain) and such targeted tES may be applied as short duration pulses. In this way, the delivered electrical stimulations can may enhance the likelihood of subsequent slow oscillations to occur, thereby improving or prolonging the person's time spent in deep (slow wave) sleep. When tES is delivered during asynchronous and/or quiescent periods, the rate and duration of subsequent slow oscillations occurring in the person's brain can increase. When tES is delivered based on detected ongoing oscillations as a trigger, tES should be phase locked with the ongoing phase oscillations by timing the delivery of the pulsed stimulation to the cortical up states. It is also noted that although K-complexes occurring in stage 2 sleep are not necessarily the same phenomenon as slow oscillations in stage 3 or 4 sleep, the determination or detection of ongoing slow oscillations as used in the present disclosure may also generally refer to the determination of ongoing K-complexes occurring in stage 2. By determining the sleep stage of the person and monitoring their brain activity for low spectral power and/or cross-channel coherence (or DUPTs) for triggering tES, deep sleep of the person may be significantly improved and enhanced. Additionally, triggering tES based on asynchronous and/or quiescent periods may further increase the rate and duration of slow oscillations. In general, the present closed-loop approach may increase or enhance the restorative state of sleep, which could advantageously improve resistance to sleep deprivation and improve cognitive performance generally.

In another aspect, an EEG system to implement the targeted sleep enhancement described herein is provided. The EEG system monitors brain wave activity of a user. In particular, the EEG system may comprise a plurality of spatially separated EEG sensors (EEG electrodes) placed on the head of the user to generate a plurality of EEG signals. It can be desirable to place these EEG electrodes on the user's skin rather than their hair. The EEG electrodes located on the subject's head are electrically coupled to an EEG signal amplifier and a processing circuit. The processing circuit is any suitable microprocessor, microcontroller, or other basic computing device that incorporates the functions of a computer's central processing unit (CPU) on an integrated circuit or, at most, a few integrated circuits. The processing circuit (e.g., computer, tablet, or mobile phone) can be programmed to process the EEG data and control stimulation delivery. The processing circuit is coupled to memory that stores machine executable instructions. When these instructions are executed by the processing circuit, the processing circuit is programmed to facilitate data processing generally, user sleep stage detection, detection of measure of low spectral power, coherence, DUPT and/or slow oscillation biomarkers, and the triggering of the stimulation. In this way, the processing circuit executes a closed-loop targeted deep sleep enhancement algorithm.

A plurality of stimulation electrodes are also placed on the user's head, such as located near the Fp1 and Fp2 locations of the forehead (according to the standard 10-20 EEG electrode configuration) and behind the ears (mastoid electrodes). In one aspect, the stimulation electrodes include four electrodes comprising two anodes and two cathodes, in which the anodes are positioned on the forehead near EEG channel locations Fp1 and Fp2 and the cathodes are positioned on ipsilateral mastoid locations relative to the associated anode of the pair of two anodes. The locations on the user's head are described relative to the conventional 10-20 system used in the art. The stimulation electrodes may be made of silver (Ag) or silver chloride (AgCI) that can be filled with conductive gel. In another aspect, eight total electrodes can be placed on the head of the subject, comprising: two placed at F3 (left frontal lobe), two placed at F4 (right frontal lobe), two placed at the left mastoid A1, and two placed at the right mastoid A2. The stimulation electrodes are attached to a neurostimulator that is configured to deliver stimulation such as a mild low intensity tES of 2 mA, for example. The neurostimulator may be a part of the processing circuit. One suitable neurostimulator is a NeuroMod16 device available from Rio Grande Neurosciences, Dayton, Ohio, to deliver five cycles of 0.75 Hz oscillating current, for example. After the user falls asleep, the EEG system may perform closed-loop monitoring of the user's brain activity. Specifically, the EEG system can monitor the user's progress through the sleep cycle/stages and gate stimulation once the user enters stage 2 or stage 3 sleep. Subsequent to the user entering stage 2 or 3 sleep, the EEG system determines measures of spectral power and coherence to trigger tES during periods of quiescent and less synchronized period of brain activity.

Periods of brain activity with lower spectral power and synchrony are experimentally demonstrated to be negatively correlated with the resulting number of slow oscillations after tES is triggered to apply in these periods, as described in further detail below. Alternatively, DUPTs or ongoing slow oscillations may be used to trigger tES after a brief latency period so that tES is delivered while there is an ongoing slow oscillation. As described above, the stimulation can be short duration pulsed tDCS. After one instance of a series of tDCS pulses is applied, the EEG system may continue to conduct closed-loop monitoring of the user's brain activity. This closed-loop monitoring may continue as long as the processing circuit determines that the user remains in NREM sleep (and particularly, NREM stage 2 or 3). In this way, the delivered electrical stimulations may enhance the likelihood of subsequent slow oscillations to occur (or neural synchrony of such oscillations), thereby increasing the amount of time in deep sleep to improve the user's sleep quality and performance.

In another aspect, such targeted enhancement of deep sleep can also be implemented by a portable EEG headwear, such as a headband. The headband can be worn on the user's head and can be a self-contained device for targeted sleep enhancement. The headband device may comprise the plurality of spatially separated EEG electrodes, stimulation electrodes, computer processing circuit (which may include the neurostimulator) and associated memory, and a battery to wirelessly power the headband. The memory may contain executable instructions for the computer processing circuit to execute a closed-loop targeted deep sleep enhancement algorithm, as described above. In this algorithm, real-time detection of sleep biomarkers (e.g., spectral power and cross-channel coherence) are leveraged to enhance deep stages of sleep associated with restoration. To this end, precise neurostimulation is applied during NREM stages of sleep to increase the duration and intensity of the user's deep sleep. Unlike an indiscriminately applied electrical stimulation (i.e., open-loop methodology), the present disclosure provides real-time or near real-time detection and delivery for precise targeting of tES based on measurements of biological phenomena in the user's brain. As such, this precisely timed neurostimulation enables a lower dose of stimulation and/or longer duration to achieve greater efficacy of deep sleep enhancement.

More generally, transitions events such as large amplitude DUPTs (e.g., exceeding a detection threshold of −50 or −80 μV) are electrical signals generated by the brain of an individual that may be detected. The stages of sleep may include specific brain waves (i.e., detectable EEG waveforms) which may be classified based on waveform frequency. One conventional frequency classification includes delta waves ranging from 1 to 4 Hertz (Hz), theta waves ranging from 5 to 7 Hz, alpha waves ranging from 8 to 11 Hz, sigma waves ranging from 12-15 Hz, beta waves ranging from 15 to 25 Hz, and gamma waves ranging from 26 to 50 Hz. Other suitable frequency classifications may also be used. Stage 2 sleep includes certain characteristic EEG waveform patterns such as sleep spindles and K complexes. Stage 3 sleep includes slow oscillations containing both a negative half wave (down state) and a positive half wave (up state). DUPTs indicate a transition from a cortical down-state to a cortical up-state, and can be detected effectively as a biomarker revealing the existence of an ongoing slow oscillation. Cortical up-states may also reflect a state of significant neural plasticity. Characteristic EEG waveform patterns can generally be referred to as biomarkers.

K-complexes are high voltage biphasic waves comprising a sharp negative wave followed by a positive high voltage slow wave. The duration of a K-complex should exceed 0.5 seconds. K-complexes may have higher amplitudes than slow oscillations. In the context of the present disclosure, determining an ongoing slow oscillation based on a detected DUPT may also refer to ongoing K-complexes. Stage 3 sleep also includes certain characteristic EEG waveform patterns such as large amplitude slow oscillations (slow waves). Slow waves or oscillations refer to waves ranging in frequency from 0.5 to 4 Hz with amplitudes exceeding 75 μV, for example. K-complexes and slow waves are both examples of phenomena containing detectable transition events. Such transition events may be detected and used as an alternative trigger for gating tES. That is, they are an alternative to using periods of quiescent and/or asynchronous activity as the trigger for delivering tES. Slow oscillatory tDCS applied during periods of low spectral power and/or coherence can effectively modulate the rate and duration of slow oscillations. This increase in rate and duration during NREM stages 2 and 3 increases the duration the subject spends in deep sleep as well as increasing memory recall for recently learned facts (which supports the proposition that slow oscillations play an active role in declarative memory consolidation). Accordingly, this closed loop sleep enhancement methodology can increase deep sleep to improve restorative deep sleep quality and performance. The closed-loop methodology refers to an approach to targeted sleep enhancement comprising delivering tES synchronously with naturally occurring periods of low power and low synchrony brain activity.

For a particular subject such as a human, the signals of interest are detected through the small set of electrodes that are configured to be applied noninvasively to the subject's scalp. These signals, such as EEG signals, are processed with a set of algorithms in real-time to achieve two functions that are necessary for the accurate and effective delivery of the intervention signal (electrical stimulation). The signals may be EEG signals and the small set of electrodes may be part of an EEG headband or system, as described above. First, a sleep stage detection algorithm is used to classify the sleep stage of the subject using, for example, a hypnogram. The sleep stage detection algorithm can monitor the subject's sleep cycle and determine that the subject is in one of NREM sleep stage 2 through 3. Second, the closed-loop targeted deep sleep enhancement algorithm is used to gate and apply the electrical stimulation. Gating can be achieved by determining NREM stage 2 or stage 3 (from the sleep stage detection algorithm) and identifying a period of quiescent and asynchronous brain activity based on measures of spectral power and coherence. In this way, tES is advantageously applied during the quiescent and asynchronous period to increase the duration and rate of slow oscillations. Alternatively, tES is applied during an ongoing slow oscillation based on using a DUPT or another suitable neurophysiological biomarker to determine the existence of the slow oscillation. In this alternative trigger closed-loop methodology, the latency between detecting DUPTs and delivery of the electrical stimulation can be minimal, such as between 50 and 200 milliseconds (ms). In either methodology, the closed loop sleep enhancement approach of the present disclosure may be applied to naps or overnight sleep, for example.

In various aspects, the apparatus, system, and method according to the present disclosure specifically uses a closed-loop methodology to specifically deliver interventions of tES at optimal times to target periods of quiescent and asynchronous brain activity. In this way, the closed-loop methodology of the present disclosure discloses short duration tES as an approach to improve memory-related sleep physiology and enhance declarative memory consolidation and learning. In particular, short duration tES delivered during NREM stage 2 or stage 3 sleep may increase the rate and duration of slow oscillations, corresponding to a greater proportion of time spent in NREM stage 3 sleep relative NREM stage 1 over a specific sleep cycle. In an alternative method, tES can be delivered to target DUPTs. Targeting DUPTs involves using tES that is phase-dependent on an individual's natural neural oscillations such that the electrical stimulation is delivered to increase the spectral amplitude and/or likelihood of synchronous activation (e.g., with respect to the natural neural oscillations) of ongoing slow oscillations in the same frequency band (e.g., delta frequency band) after detecting a DUPT. Moreover, the present disclosure includes the function of performing a complete classification of sleep stages so that tES may be applied to the subject during NREM stage 2 or stage 3 sleep.

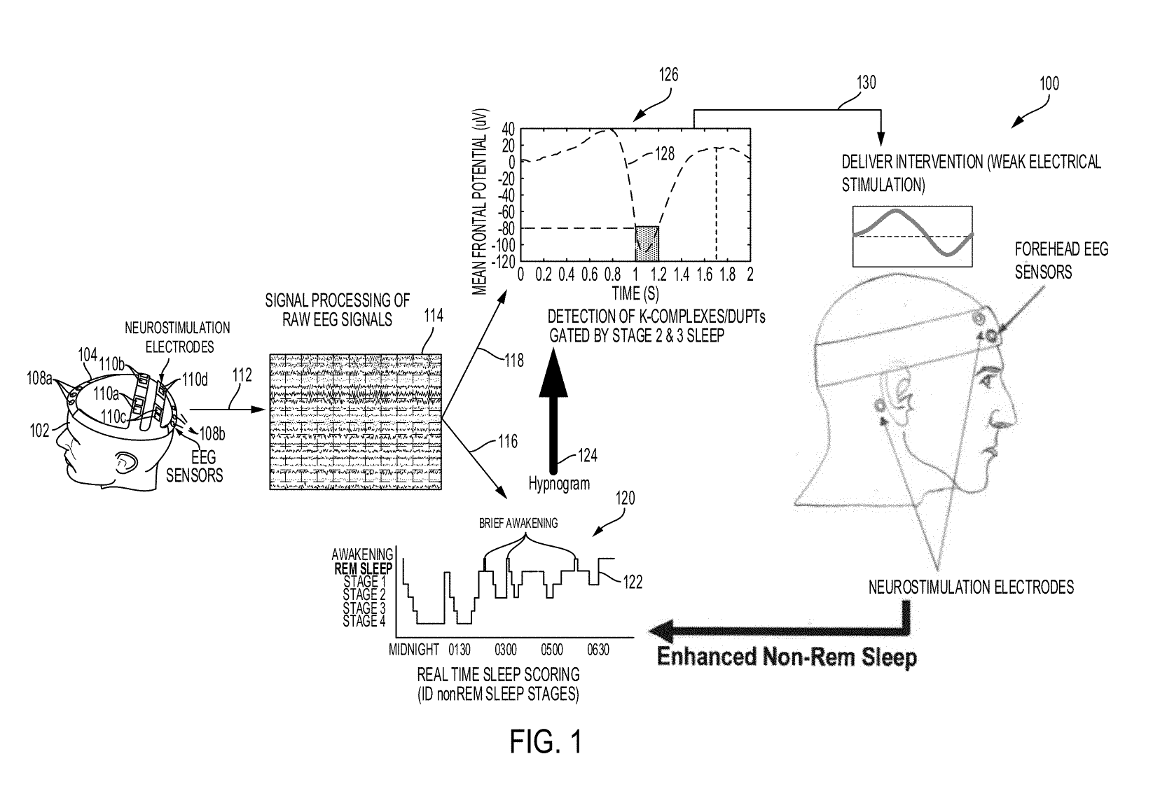

FIG. 1 is a diagram 100 that represents the closed-loop operation of a targeted deep sleep enhancement system according to one aspect of the present disclosure. In one aspect, the process includes sensing activity in the cerebral cortex of a subject 102 using EEG. The subject 102 wears an EEG headset 104 having a relatively high density set of EEG electrodes that cover the surface of the scalp. The EEG electrodes may be arranged in a 32-channel system, for example. The headset 104 comprises a plurality of EEG sensors 108a, 108b. The headset 104 may be a suitable EEG electrode system such as a Brain Products 32-channel antiCAP electrode system and BrainAMP DC amplifier of Brain Products, GmbH, Munich, Germany in the standard 10-20 electrode layout, for example. Throughout this disclosure, the EEG sensors also may be alternatively referred to as EEG electrodes or EEG channels. A first set of EEG sensors 108a is placed near the frontal lobe of the cerebral cortex and a second set of EEG sensors 108b is placed near the occipital lobe of the cerebral cortex. In another aspect, only the first set of EEG sensors 108a are used such that the headset 104 comprises only the EEG sensors 108a positioned on the forehead of the subject 102. This enables the EEG sensors 108a only to contact the head of the subject 102 in areas where there is no hair. It may be desirable not to place EEG sensors on the subject's 102 hair to avoid potential problems associated with processing EEG signals received through hair.

In aspects where only the first set of EEG sensors 108a are provided such that only brain activity near frontal channels (corresponding to areas near the frontal lobe) is detected, executing the sleep stage detection algorithm may not involve alpha wave activity. In fact, measurements of alpha wave activity may not be prominent on frontal channels. The headset 104 also comprises sets of neurostimulation electrodes 110a, 110b, 110c, 110d for applying an electrical stimulation intervention signal. Each of the neurostimulation electrode sets 110a-d may comprise three electrodes or some other suitable number of electrodes. The sets of neurostimulation electrodes 110a-110d may be positioned at the front of the head of the subject 10 so as to avoid contacting hair (similar to EEG sensors 108a). Two additional neurostimulation electrodes may be placed behind the ears, on the mastoid regions of the subject 102, for example. The two additional neurostimulation electrodes can function as the cathodes for the neurostimulation electrode sets 110a-110d. In another aspect, the neurostimulation electrodes 110a-110d are configured so that electrodes 110a-110b are anodes and electrodes 110c-110d are cathodes. In the conventional 10-20 system, the anodes 110a-110b may be positioned at the Fp1 and Fp2 locations while the cathodes 110c-110d are positioned at the mastoid locations. Specifically, each cathode 110c-110d can be ipsilateral relative to the anodes 110a-110b (e.g., Fp1 and A1 on one side and Fp2 and A2 on the other side). Reference electrodes may also be attached to the left and right mastoid sites of the subject with an adhesive ring, for example.

In various aspects, additional or fewer EEG electrodes 108a-108b or neurostimulation electrodes 110a-d may be employed without departing from the scope of the present disclosure. In one aspect, three EEG sensors 108a are placed over the frontal lobe of the cerebral cortex for implementation of the sleep stage detection algorithm. EEG sensors 108b placed over the occipital lobe of the cerebral cortex may be unnecessary for the sleep stage detection and transition events detection algorithms. However, in alternative aspects, a suitable number of occipital EEG sensors 108b, such as three, are positioned over the occipital lobe.

While the subject 102 sleeps, EEG signals 112 can be sensed by the EEG sensors 108a-b based on electrical activity from the brain of the subject 102. Once the signals 112 are collected, the signals 112 are provided to a digital signal processing circuit to process the raw EEG signals 112. The computer processing circuit described above may comprise the digital signal processing circuit. Based on the collected EEG signals 112, the digital signal processing circuit produces digital EEG waveforms 114 for subsequent analysis. The computer processing circuit is configured to execute or implement specific algorithms for this analysis. Specifically, in one aspect, the computer processing circuit implements and executes the sleep stage detection algorithm and closed-loop targeted deep sleep enhancement algorithm described above. The sleep stage detection algorithm could also be considered part of the deep sleep enhancement algorithm. The sleep stage detection algorithm comprises decoding the digital EEG waveforms 114 for determination of what stage of sleep the subject 102 is in.

The present disclosure classifies stages of sleep into stages 1 through 3 (NREM sleep) and REM sleep. However, other suitable classifications may also be used including ones in which the sleep stages include NREM stage 4. Those skilled in the art will appreciate that NREM sleep can be further classified into light and deep sleep. These light and deep sleep stages can be labeled according to either the classification into stages 1 to 3 or into stages 1 to 4. As described herein, the present disclosure may provide for enhancing the duration of deep sleep. Each sleep stage can be characterized by unique waveforms and patterns that describe the activity in the brain of the subject 102. As described above, it can be necessary to know which stage of sleep the subject 102 is in for targeted sleep enhancement.

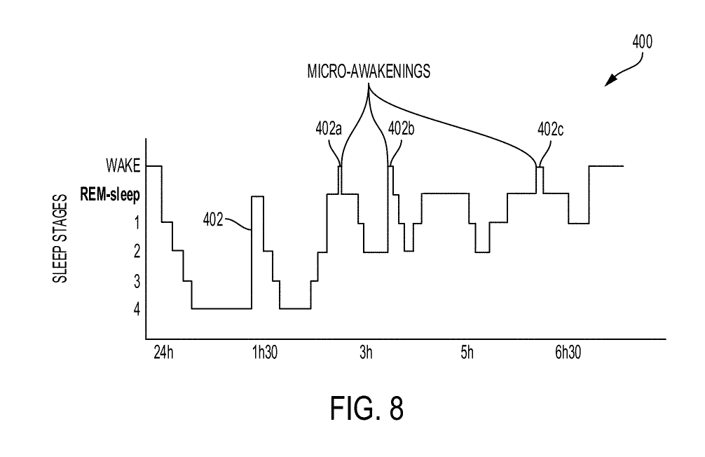

The sleep stage detection algorithm 116 may effectively detect the sleep stage of the subject 102 in real time or near real time. In one aspect, the sleep stage detection algorithm 116 involves generating a hypnogram 120 which comprises a graph 122 to represent the stages of sleep as a function of time. Polysomnography techniques are used to generate the hypnogram 120. Specifically, EEG, electrooculogram (EOG), and electromyography (EMG) measurements can be used to score or classify a polysomnographic record of sleep into sleep stages. The sleep scoring or staging could occur automatically. Such sleep stages are depicted in the hypnogram 120 and in more detail with respect to the hypnogram 400 shown in FIG. 8. As shown in the hypnogram 120 in FIG. 1 and described in more detail with reference to the hypnogram 400, the stages of sleep are provided along the vertical axis and time is provided along the horizontal axis. The hypnogram 120, 400 provides an easy way to present the recordings of brain wave activity from a digital EEG waveform 114 during a period of sleep according to the sleep stage classification of the present disclosure. The wake state, REM sleep, and NREM sleep (comprising stages 1 to 4) of the subject 102 are determined and displayed on the hypnogram 120, 400, as indicated along the vertical axis of the hypnogram 120, 400. As previously described, NREM sleep can be classified into three stages rather than the four stages depicted in hypnogram 120, 400. In such three stage NREM classifications, the 4th stage of NREM sleep may be subsumed within stage 3. Furthermore, stage three as defined in three stage NREM classifications can be labeled slow wave sleep (SWS) and is the deepest stage of sleep. The duration of stage 3 SWS can be augmented by the deep sleep enhancement algorithm, as described herein.

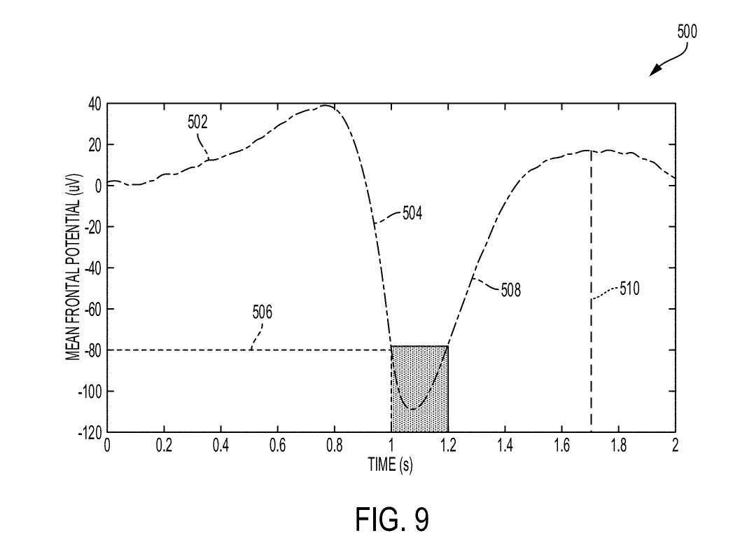

Upon detecting the sleep stage of the subject 102, the sleep stage detection algorithm 116 triggers or gates 124 the deep sleep enhancement algorithm 118 to detect a specific signal 126 of interest in the digital EEG waveform 114. In one aspect, the signal 126 of interest comprises a down-state to up-state transition event (DUPT) 128. Upon detecting the DUPT 128, the deep sleep enhancement algorithm 118 may deliver an intervention signal 130 to the subject 102. Detection of the DUPT signal 128 is further described with reference to FIG. 9. As described in further detail below, the DUPT signal 128 is an alternative trigger to measures of spectral power and/or coherence exceeding thresholds to indicate periods of low power and/or synchrony as triggers for the intervention signal 130. In either case, the intervention signal 130 may be a transcranial weak electrical stimulus conducted through the neurostimulation electrodes 110a-d to target ongoing slow oscillations. Certain parameters of the electrical stimulation, such as the frequency (which may be specific to the subject 102) and the duration of electrical current delivery can be specified. The frequency can be based on detected slow oscillations and monitoring the response of the brain to each instance of electrical stimulation. The duration can be 4 seconds of pulsed tDCS at 1 Hz, for example. A pulsed tDCS waveform includes certain specified pulse characteristics such as rise, plateau, and fall time. When specifying the parameters of stimulation it may be beneficial to link them as closely as possible to the natural characteristics of the individual's neural oscillations.

When the subject 102 is asleep, the brain of the subject 102 oscillates between states of excitability and states of less excitability. In other words, the brain oscillates between cortical up-states and cortical down-states respectively. Detecting DUPT signals 128 enables gating of the electrical stimulation 130 so that the electrical stimulation 130 can be delivered synchronously with determined ongoing slow oscillationss. To this end, it may be desirable to set the delivery time of the electrical stimulation 130 at a time that is close to the crossing from the cortical down-state to up-state (depicted in FIG. 9). When the delivery of the electrical stimulation 130 is timed to enhance a slow oscillation by increasing the spectral amplitude of the slow oscillation, the depth of sleep may be enhanced. Moreover, the targeted electrical stimulation 130 may increase or enhance the restorative state of sleep, which could advantageously improve recovery from sleep deprivation and improve performance generally. For example, the delivered electrical stimulation 130 may increase the duration that the subject 102 spends in deep sleep.

The down-state to up-state transition event 128 can be very robust and readily detected in stage 2 through 4 sleep. As described above, in stage 2 sleep, the high voltage biphasic waves which include DUPTs 128 are called K-complexes. It should be understood that all k-complexes contain down-state to up-state transition events 128, but not all down-state to up-state transition events 128 are K-complexes. Similarly, the large amplitude waves which occur in stage 3 sleep are called slow oscillations or slow waves. Slow waves also include transition events 128. However, as described above, determining ongoing slow oscillations as used herein can also refer to determining ongoing K-complexes. Stage three sleep can be defined as a deep stage of sleep in which the brain is engaged in very slow rhythmic activity. During NREM stage 2 through 3 sleep, the deep sleep enhancement algorithm 118 may determine that the brain of the subject 102 exhibits a sufficiently large oscillation that constitutes a sufficiently large change in excitability to qualify as a DUPT 128.

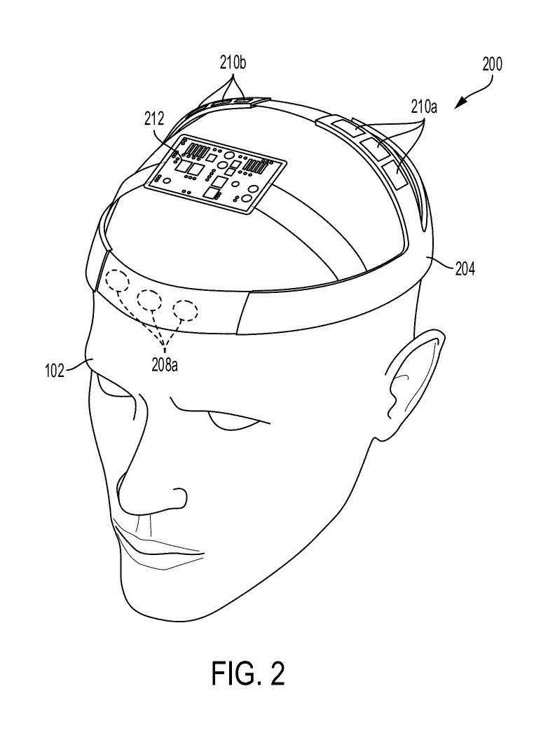

FIGS. 2-6 illustrate various views of hardware components of a targeted deep sleep enhancement system for monitoring brain activity and stimulating ongoing slow oscillations in the brain during NREM stage 2 through stage 3 sleep according to various aspects of the present disclosure. The targeted deep sleep enhancement system 200 depicted in FIGS. 2-6 includes a subject 102 wearing a headset 204 comprising a first and second plurality of spatially separated EEG sensors 208a, 208b, neurostimulation electrode sets 210a, 210b, 210c, 210d, and a computer processing circuit 212. Each of the first and second plurality of spatially separated EEG sensors 208a-b, neurostimulation electrode sets 210-210d, and computer processing circuit 212 may be positioned on or embedded in bands of the headset 204, as illustrated in FIGS. 2-6.

The computer processing circuit 212 comprises a computing system having one or more microprocessor(s), microcontroller(s), digital signal processor, memory, input/output (I/O), analog-to-digital converter (ADC) circuits, digital-to-analog converter (DAC) circuits, and other features required of a functional computer to process EEG signals, execute the sleep stage detection algorithm 116 (FIG. 1), deep sleep enhancement algorithm 118 (FIG. 1), and deliver the intervention signal 130, among other functions. The computer processing circuit 212 may include a computer, tablet, or mobile phone, for example. The computer processing circuit 212 may be coupled to a memory containing machine executable instructions for executing of the functions and algorithms of the deep sleep enhancement methodology described herein. A neurostimulator of the computer processing circuit 212 can be configured to deliver mild electrical stimulation such as pulsed tDCS at 2 mA. The computer processing circuit 212 receives inputs comprising EEG signals from the EEG sensors 208a-b, processes these inputs, and converts them into digital EEG waveforms that may be employed by the sleep stage detection algorithm 116 and the deep sleep enhancement algorithm 118.

In one aspect, the spatially separated EEG sensors 208a, 208b of the sleep enhancement system 200 are configured to sense activity in the cerebral cortex of a subject 102 using EEG. As shown in FIGS. 2-6, the subject 102 wears the headset 204 having a relatively high density set of EEG electrodes that cover the surface of the scalp. The components of the headset may be arranged relative to the cerebral cortex of the subject 102. The neurostimulation electrodes 210a-d may be positioned near the occipital lobe of the cerebral cortex. Each of the neurostimulation electrode sets 210a-210d may comprise three electrodes or some other suitable number of electrodes. The sets of neurostimulation electrodes 210a-210d can also be positioned on another area of the head of the subject 102, or not provided at all. The computer processing circuit 212 may be positioned near the central sulcus or parietal lobe of cerebral cortex. The first plurality of spatially separated EEG sensors 208a may be placed near the frontal lobe of the cerebral cortex and the second plurality of spatially separated EEG sensors 208b may be placed near the occipital lobe of the cerebral cortex. In alternative aspects, only the first plurality of spatially separated EEG sensors 208a is provided in the targeted sleep enhancement system 200. The sleep stage detection algorithm 116, deep sleep enhancement algorithm 118 and other functions of the targeted sleep enhancement system 200 may only require EEG signals from the first plurality of spatially separated EEG sensors 208a. The first plurality of spatially separated EEG sensors 208a may be configured to be positioned such that they contact only portions of the forehead or head of the subject 102 where there is no hair.

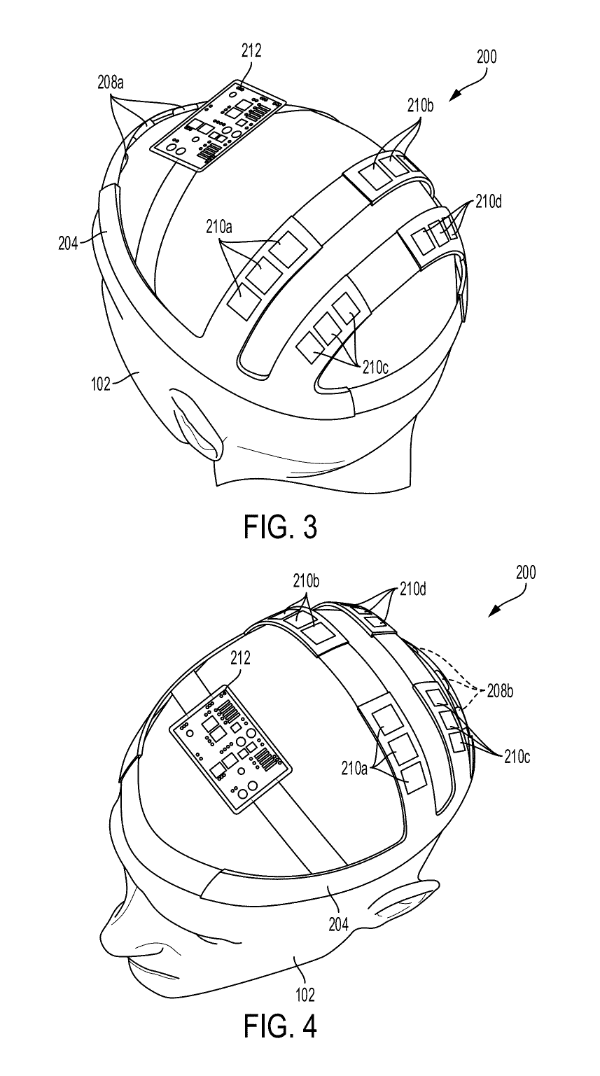

In various aspects, additional or fewer EEG electrodes 208a-208b or neurostimulation electrodes 210a-210d may be employed without departing from the scope of the present disclosure. FIGS. 2-3, for example, depict that the first plurality of spatially separated EEG sensors 208a comprises three EEG sensors positioned over the frontal lobe. In FIGS. 2-3, no second plurality of spatially separated EEG sensors 208b is positioned over the occipital lobe. As shown in FIG. 3, the first plurality of spatially separated EEG sensors 208a are placed on the interior side of the headset 204. The first plurality of spatially separated EEG sensors 208a may also be positioned in other suitable configurations or locations proximal to the frontal lobe. FIG. 2 is a frontal view of the sleep enhancement system 200 comprising the subject 102 wearing the headset 204 described above. In FIG. 2, the first plurality of spatially separated EEG sensors 208a are shown in dashed line. Additionally, in FIG. 2, only the sets of neurostimulation electrodes 210a-210b are shown.

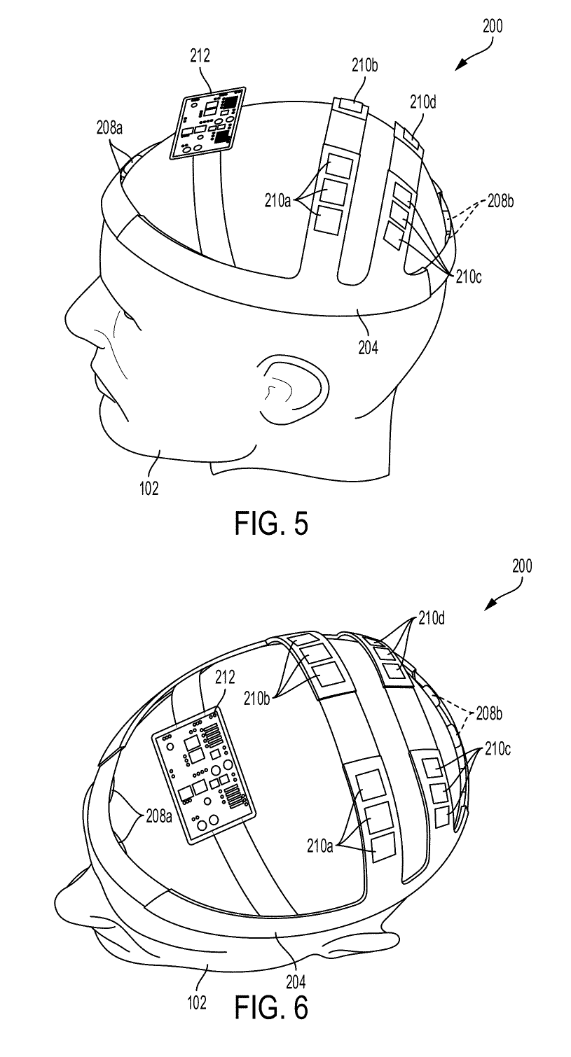

FIG. 3 is an occipital view of the sleep enhancement system 200 comprising the subject 102 wearing the headset 204 described above. In FIG. 3, the first plurality of spatially separated EEG sensors 208a is visible and positioned on the interior side of the headset 204. Also in FIG. 3, the second plurality of spatially separated EEG sensors 208b is not provided. FIG. 3 further depicts the sets of neurostimulation electrodes 210a-210d. FIG. 4 is a side view of the sleep enhancement system 200 comprising the subject 102 wearing the headset 204 described above. In FIG. 4, the second plurality of spatially separated EEG sensors 208b is shown in dashed line. FIG. 5 is another side view of sleep enhancement system 200 comprising the subject 102 wearing the headset 204 described above. In FIG. 5, the first plurality of spatially separated EEG sensors 208a comprises two EEG sensors and the second plurality of spatially separated EEG sensors 208b is shown in dashed line. FIG. 6 is yet another side view of the sleep enhancement system 200 comprising the subject 102 wearing the headset 204 described above. In FIG. 6, the first plurality of spatially separated EEG sensors 208a comprises two EEG sensors and the second plurality of spatially separated EEG sensors 208b are also shown in dashed line.

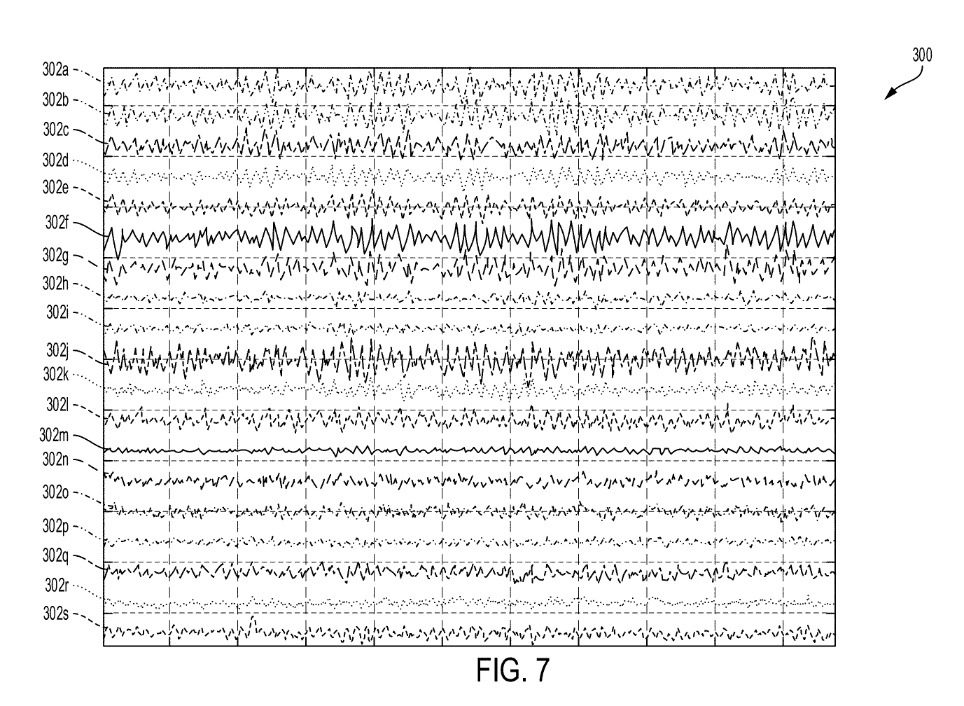

Referring now to FIGS. 1-6, in one aspect, the EEG signals 112 (FIG. 1) are sensed by the EEG sensors 208a-208b while the subject 102 sleeps. Once the raw EEG signals 112 are collected, they are provided to the digital signal processor of the computer processing circuit 212 where the raw EEG signals 112 are processed to produce digital EEG waveforms 114 (FIG. 1). The digital EEG waveforms 114 (as described further with reference to the plurality of digital EEG waveforms 300 depicted in FIG. 7) are provided to the sleep stage detection algorithm 116 to decode the digital EEG waveforms 114 and determine the stage of sleep of the subject 102 Upon detecting the stage of sleep of the subject 102, the deep sleep enhancement algorithm 118 detects a DUPT transition event 128 (FIG. 1), as further described with reference to the graphical depiction 500 of a specific EEG signal 502 of interest depicted in FIG. 9. Upon detecting the DUPT 128, the computer processing circuit 212 determines the existence of an ongoing slow oscillation and delivers an intervention signal 130 to the subject 102 via the neurostimulation electrodes 210a-d. The intervention signal may be applied to the neurostimulation electrodes 210a-d individually or selectively.

FIG. 7 is a diagram of a plurality of digital EEG waveforms 300 that are sensed by a plurality of EEG sensors according to various aspects of the present disclosure. With reference to FIGS. 1-7, each of the first and second plurality of spatially separated EEG sensors 208a-208b (in the headset 204 worn by the subject 102) detects the raw EEG signals 302a-302s while the subject 102 sleeps. Each raw EEG signal 302a-302s may correspond to a channel of the EEG sensors. The raw EEG signals 302a-302s can be amplified by an analog amplifier for further processing by the computer processing circuit 212. The amplified raw EEG signals 302a-302s may be converted by an ADC circuit to digital EEG signals and processed by the computer processing circuit 212 to produce the plurality of digital EEG waveforms 300. The plurality of digital EEG waveforms 300 are provided first to the sleep stage detection algorithm 116 to decode the digital EEG waveforms and determine the subject's 102 stage of sleep. Upon detecting the sleep stage of the subject 102, the digital EEG waveforms 300 are provided to the deep sleep enhancement algorithm 118 to detect a DUPT 128 in a specific signal 126 of interest. Upon detection of the DPT 128, the deep sleep enhancement algorithm 118 delivers a tES 130 to the subject 102 via the neurostimulation electrodes 210a-d to amplify the likelihood of synchronous activation of ongoing slow oscillations while the subject 102 is in sleep stage 2 through 3. This may improve the duration and intensity of deep sleep for the subject 102. After applying one instance of a pulsed tDCS, the deep sleep enhancement algorithm 118 and sleep stage detection algorithm 600 may continue to monitor the subject 102 for NREM sleep stage 2 and 3, DUPTs 128, and ongoing slow oscillations in those sleep stages.

FIG. 8 depicts a hypnogram 400 to determine the stages of sleep of a subject 102 according to various aspects of the present disclosure. The sleep stages are provided along the vertical axis and time is provided along the horizontal axis. As shown in FIG. 8, the sleep stages include wake stage, REM sleep stage, stage 1, stage 2, stage 3, and stage 4. As discussed above, stages 1 through 4 are considered NREM sleep. Other suitable classifications, such as a classification with only three stages of sleep, may also be used by the sleep enhancement system 200. Time is indicated at specific discrete points, including 24 hours, 1.5 hours, 3 hours, 5 hours, and 6.5 hours. The sleep stage detection algorithm 116 of the present disclosure detects in real time the stage of sleep of a subject. In one aspect, the sleep stage detection algorithm 116 generates the hypnogram 400 using a suitable polysomnography technique. A graph 402 represents the stages of sleep as a function of time.

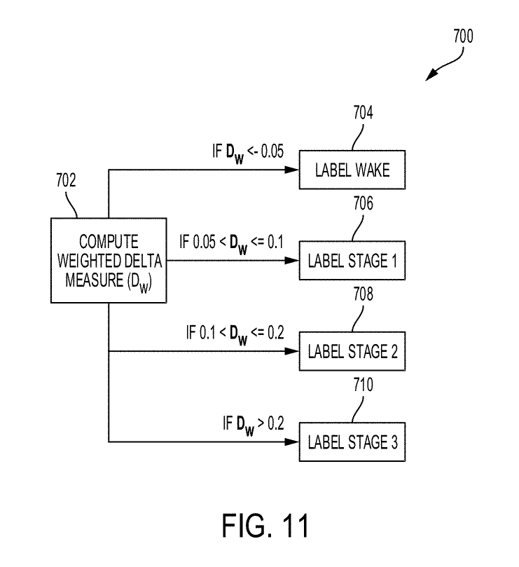

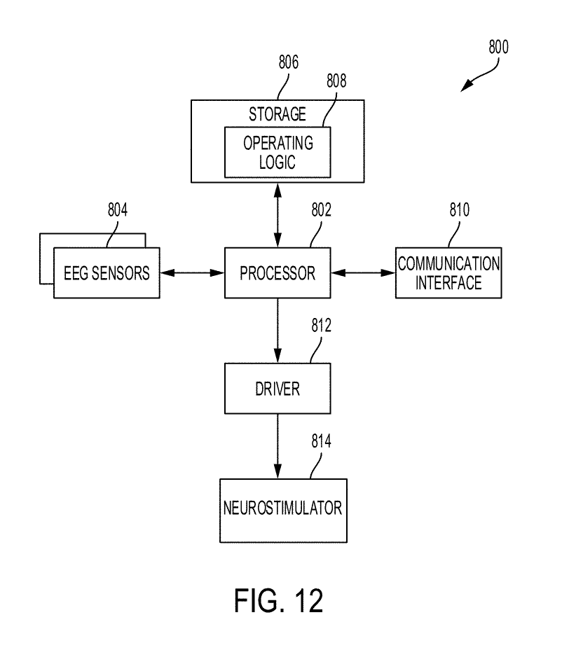

The hypnogram 400 provides an easy way to present the recordings of brain wave activity from a digital EEG waveform 114 during a period of sleep. The graph 402 allows the different REM and NREM sleep to be identified during the sleep cycle of a subject. The transitions between various sleep stages may be identified as a function of time using the graph 402. Moreover, as shown in the hypnogram 400, the periods of micro-awakenings 402a, 402b, 402c are readily detectable. Micro-awakenings can occur during the period between the REM sleep state and the wake state. To generate the hypnogram 400, the sleep stage detection algorithm 116 may involve monitoring parameters such as EEG, EOG, EMG as well as cardiopulmonary parameters such as electrocardiogram (ECG) and air flow. These monitored parameters may be used to score or classify the sleep stages as a function of time. For example, a decreased tonic component in an EMG signal may be an indication of REM sleep. Additionally or alternatively, a computed weighted delta measure may be used for sleep stage scoring, as described in further detail in connection with FIGS. 11 and 12.

FIG. 9 is a graphical depiction 500 of a specific EEG signal 502 of interest in a digital EEG waveform according to various aspects of the present disclosure. The vertical axis represents mean frontal potential (μV) and the horizontal axis represents time (s). As shown in FIG. 9, the mean frontal potential ranges from −120 to 40 μV and may correspond to EEG signals sensed by the first plurality of spatially separated EEG sensors 208a positioned near the frontal lobe. The time ranges from 0 seconds to 2 seconds. In one aspect, the specific EEG signal 502 of interest comprises a large amplitude DUPT 128, which may be detected by the deep sleep enhancement algorithm 118. The EEG signal 502 of interest has a negative edge 504 transition that indicates a steep potential voltage drop. As shown in FIG. 9, the negative edge 504 starts at approximately 0.8 seconds from a high potential of approximately 40 μV and ends at a low potential of approximately −110 μV. The deep sleep enhancement algorithm 118 may be configured to monitor a threshold mean frontal potential value for detection of a large amplitude transition event. When the EEG signal 502 drops below a predetermined large amplitude threshold 506, the deep sleep enhancement algorithm 118 determines that a DUPT 128 event has occurred.

Although the predetermined threshold potential 506 is −80 μV in FIG. 9, other suitable thresholds such as −50 μV may also be used. The threshold 506 may be empirically actively defined on the subject of a study by determining the variation in the signal 502 itself. For example, if a subject has a thick scalp or a thick skull that produces EEG signals 502 which are weaker than average, the deep sleep enhancement algorithm 118 may adjust the threshold 506 dynamically to compensate for the weaker EEG signals. After the EEG signal 502 drops below the threshold 506, it begins to rise again as indicated by the rising edge 508. As shown in FIG. 9, the rising edge 508 starts at approximately −110 μV and ends at the peak potential 510. The peak potential 510 is approximately 15 μV at 1.7 seconds. The delivery of an intervention signal 130 may further increase the amplitude of the peak potential 510 and/or the likelihood of synchronous activation. In contrast, the negative edge 504 can correspond to a polarized cortical down-state.

In another aspect, the computer processing circuit 212 may be configured to determine that the detection of a transition event is a false positive. When the deep sleep enhancement algorithm 118 detects that the negative edge 504 transition of the specific EEG signal 502 has crossed the −80 μV threshold 506, the deep sleep enhancement algorithm 118 determines whether the negative edge 504 transition of the specific EEG signal 502 initiated from a positive potential in order to mitigate false positives that, for example, can be due to drift in the EEG electrodes. In other words, a false positive may comprise the EEG signal 502 meandering aimlessly towards the negative potential because of bad grounding or some other similar reason. Thus, the false positive detection of the deep sleep enhancement algorithm 118 may reduce or prevent erroneously labeling the negative edge 504 transition of the EEG signal 502 a down-state to up-state transition event when the EEG signal 502 is a bad signal. Once the deep sleep enhancement algorithm 118 determines that the negative edge 504 transition crossed the threshold 506 within a narrow window of time and started from a positive potential, the deep sleep enhancement algorithm 118 determines that a down-state to up-state transition event is occurring. The narrow window of time may be, for example, about 400 milliseconds (ms) which starts 400 ms before the −80 μV threshold 506 crossing. There may also be a latency period between the detection of the transition event before the intervention signal 130 is delivered. The latency period may be between 50 to 200 milliseconds (ms), such as 80 ms.

The determination by the deep sleep enhancement algorithm 118 that the EEG signal 502 initiated from a positive potential can indicate that there was a very sharp negative deflection in the specific EEG signal 502. This type of sharp negative deflection occurring during sleep stages 2 through 3 is consistently associated with a DUPT 128. Accordingly, in sleep stage 2 through 3, the deep sleep enhancement algorithm 118 is configured to detect exceptionally large transitions or slow oscillations of the EEG signal 502. Thus, the sleep stage detection algorithm 116 may determine a subject is in sleep stage 2 through 3 before the deep sleep enhancement algorithm 118 detects a DUPT 128, determines the presence of an ongoing slow oscillation based on the detected DUPT, and delivers a tES 130 to the slow oscillation. Referring back to FIGS. 2-6, the deep sleep enhancement algorithm 118 may detect transition events based on taking a spatial average of the first plurality of spatially separated frontal EEG channels 208a and determining a large negative change in mean frontal potential as shown in FIG. 9.

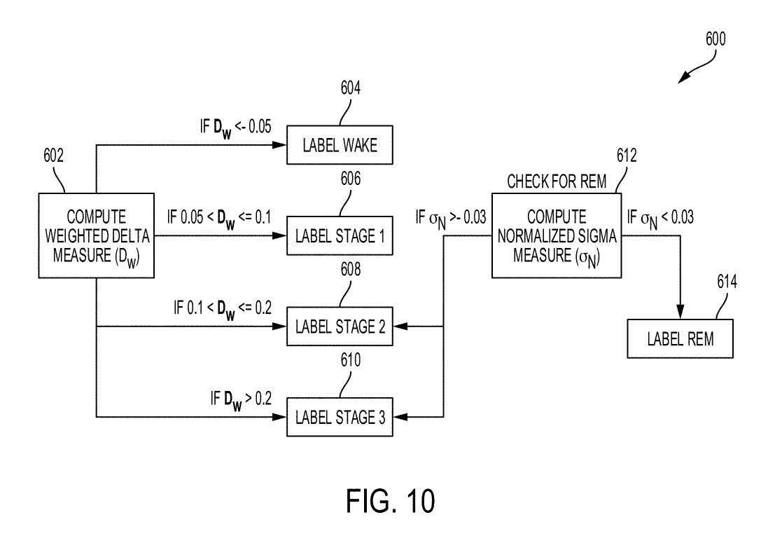

FIG. 10 is a flow diagram of a sleep stage detection algorithm 600 (which is similar to the sleep stage detection algorithm 116) according to various aspects of the present disclosure. The sleep stage detection algorithm 600 may employ high density EEG to enable reduction of noise through spatial averaging. Such noise reduction results in stronger EEG features sampled for better distinguishing differences in detected brain wave activity from different regions of the brain across sleep stages. In other words, noise reduction can enable the sensed EEG signals from the frontal channels corresponding to the first plurality of spatially separated EEG sensors 208a and the occipital channels corresponding to the second plurality of spatially separated EEG sensors 208b to be easily distinguishable. Additionally, the sensed EEG signals from the individual channels comprising the frontal channels and the occipital channels should be easily distinguishable. The frontal channels can comprise EEG sensors labeled Fp1, Fp_{z} and Fp2 (according to the 10-20 EEG system). The occipital channels can comprise EEG sensors labeled Oz, O1, O2. In one aspect, the sleep stage detection algorithm 600 determines two spectral features for sleep stage classification. The first spectral feature is called a weighted delta measure (D_{w}) for distinguishing between sleep stages 1 through 3. The second spectral feature comprises a multiplication (μ_{N}·σ_{N}) between normalized mean sigma power (μ_{N}) and variance in sigma power (σ_{N}) for distinguishing between REM and NREM stages of sleep.

The second spectral feature μ_{N}·σ_{N} may be calculated via measurements from central channels, which correspond to EEG sensors labeled Cz, C3, C4 that are positioned proximal to the central portion of the brain (i.e. the central sulcus or parietal lobe). EEG sensors Cz, C3 and C4 are not shown in FIGS. 1-6. The normalized variance sigma power σ_{N} may also be used in isolation for distinguishing between REM and NREM stages of sleep. A threshold of 0.03 may be used such that if the σ_{N} value is calculated to be less than the 0.03 threshold, the subject 102 is in a REM sleep stage. Furthermore, if the σ_{N} value is calculated to be equal to or greater than the 0.03 threshold, the subject 102 may be determined to be in a NREM stage of sleep, such as sleep stage 2 or 3. In both determinations of the first and second spectral features, the sleep stages are computed in 5 second time epochs and smoothed with a conservative approach. The deep sleep enhancement algorithm 118 searches for DUPTs 128, as described above. Epochs containing arousals (high power across all EEG channels, which correspond to high power across all waveform frequency bands) are labeled as wake stage.

As shown in FIG. 10, in one aspect, the sleep stage detection algorithm 600 computes 602 the weighted delta measure D_{w}. D_{w} can be calculated based on alpha, delta, and gamma power measurements from three frontal channels and three occipital channels. As described above, the headset 204 may include three frontal EEG sensors (channels Fp1, Fpz, and Fp2) and three occipital EEG sensors (channels Oz, O1, and O2) for a total of 6 EEG channels. The three frontal EEG sensors form the first plurality of spatially separated EEG sensors 208a and the three occipital EEG sensors form the second plurality of spatially separated EEG sensors 208b. In this aspect, the weighted delta measure D_{w} may be calculated as the Delta Alpha Gamma ratio (DAG), where:

$${D}_{w} = \frac{D}{A * G}$$

D=weighted delta power (0-4 Hz) over 3 frontal EEG channels
A=alpha power (8-13 Hz) over 3 occipital (back of head) EEG channels
G=gamma power (30-40 Hz) over all 6 EEG channels

In another aspect, as discussed above, no occipital EEG sensors or channels are provided. As discussed above, alpha wave activity may not be prominent on frontal channels. Accordingly, in this aspect, D_{w} can be calculated based only on delta and gamma power measurements from the three frontal channels (Fp1, Fpz, Fp2). Accordingly, D_{w} may be calculated as the Delta Gamma ratio (DG), where:

$${D}_{w} = \frac{D}{G}$$

D=weighted delta power (0-4 Hz) over 3 frontal EEG channels
G=gamma power (30-40 Hz) over the 3 frontal EEG channels
   As discussed above, additional or fewer EEG channels may be provided, as appropriate, for calculation of the weighted delta measure D_{w} without departing from the spirit of the present disclosure. In sum, wake and all NREM stages can be classified or scored by the normalized measure D_{w} (DAG) which is computed by dividing the weighted frontal delta power (Fp1, Fpz, Fp2) by the product of the mean occipital alpha power (Oz, O1, O2) and the mean total gamma power (all EEG channels). Alternatively, wake and NREM stages can be classified or scored by D_{w} (DA), computed by dividing the weighted frontal delta power (Fp1, Fpz, Fp2) by the mean total gamma power (all EEG channels). The sleep stage detection algorithm 600 can also compute the DAG ratio using only frontal electrodes (with delta, alpha, and gamma computed from the same electrodes) with similar accuracy relative to an aspect in which both frontal and occipital electrodes are used.

The DG or DAG is computed by taking a spectral power measure of the weighted delta power, where delta corresponds to EEG signals having very low frequencies in the range of 0 to 4 Hertz. After receiving the EEG signals from the three frontal EEG electrodes, the sleep stage detection algorithm 600 can compute the delta power from EEG signals between 0-4 Hz and segment the delta power measurement down into four separate frequencies (one, two, three, and four). This segmentation is done because the lower the frequency, the more likely that the subject 102 is in a deep stage of sleep. When the frequencies are segmented, a different weighting coefficient is applied to each one of the four sleep stages for calculation of the weighted delta measure. In the DG or DAG ratio, the alpha measure may be based on a measure of occipital alpha power (EEG signals between 8-12 Hz), determined based on the occipital EEG sensors placed at the back of the head. As the subject 102 transitions from a wake state to a sleep state, occipital alpha power decreases significantly. As described above, the global gamma measure (EEG signals having frequencies between 25-50 Hz) is computed across all EEG electrodes provided on the headset 204. Gamma is a rare frequency during sleep and is more commonly associated with rapid firing rates in brain cells during wake states.

As shown in FIG. 10, once the weighted delta measure D_{w} is calculated 602, if D_{w} is ≤−0.05, the sleep stage is labeled 604 wake. If 0.05<D_{w}<=0.1, the sleep stage is labeled 606 stage 1. If 0.1<D_{w}≤0.2, the sleep stage is labeled 608 stage 2. If D_{w}>0.2, the sleep stage is labeled 610 stage 3. In sum, the D_{w} thresholds for scoring sleep stages may be applied as follows: D_{w} values less than or equal to 0.05 are classified as wake stage, D_{w} values greater than 0.05 and less than or equal to 0.1 are classified as stage 1, D_{w} values greater than 0.1 and less than or equal to 0.2 are classified as stage 2, D_{w} values greater than 0.2 are classified as stage 3. When a four stage NREM classification is used, stage 4 may also be classified based on D_{w} values greater than at least 0.2. The D_{w} thresholds used here have been empirically determined as suitable, but other suitable thresholds may be used as appropriate. As described above, the sleep stage detection algorithm 600 may also classify REM sleep by computing 612 the normalized sigma measure σ_{N}. If σ_{N}≥0.03, the sleep stage detection algorithm 600 determines that the subject 102 is a NREM stage and further determines whether the sleep stage is 2 or 3. If σ_{N}<0.03, the sleep stage of the subject 102 is labeled 614 as REM stage.

It is worthy to note that the DUPTs 128 of interest do not usually occur in REM stage sleep. Therefore, the consequence of a false positive from the standpoint of mislabeling a stage of sleep when it is actually REM is near zero and no intervention signal 130 will be triggered. Accordingly, FIG. 11 is a flow diagram of a sleep stage detection algorithm 700 (similar to sleep stage detection algorithm 116 and 600) that does not check for REM sleep stage according to one aspect of the present disclosure. Accordingly, as shown in FIG. 11, the sleep stage of the subject 102 may be classified or scored only based on the weighted delta measure D_{w}, without any consideration of the normalized sigma measure σ_{N}. Thus, once the weighted delta measure D_{w} is calculated 702, the D_{w} thresholds as described in connection with FIG. 10 can be applied. Specifically, if D_{w} is ≤−0.05 the sleep stage is labeled 704 wake. If 0.05<D_{w}≤0.1, the sleep stage is labeled 706 stage 1. If 0.1<D_{w}≤0.2 the sleep stage is labeled 708 stage 2. If D_{w}>0.2 the sleep stage is labeled 710 stage 3. In another aspect, the sleep stage detection algorithm 600 can label or classify sleep stages based on electrocardiogram (ECG) and electroculogram (EOG) electrodes. The ECG and EOG electrodes can be in addition to or a subset of the EEG electrodes on EEG headset 104. Electrophysiological data can be collected at a sample rate such as 500 Hz for offline analysis to conduct sleep scoring (as compared for reference to automatic sleep scoring performed based on D_{w}, for example). For the EOG measurements, one electrode can be placed one centimeter (cm) above the corner of the right eye and another electrode placed one cm above the corner of the left eye.

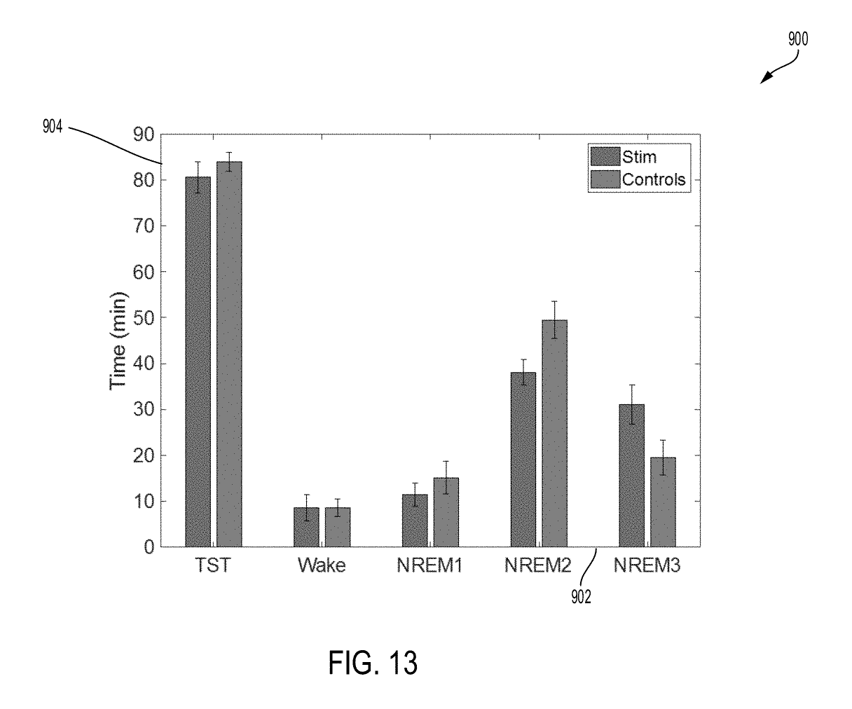

FIG. 13 is a graph 900 illustrating a duration spent in each sleep stage according to one aspect of the present disclosure. Graph 900 illustrates that closed-loop delivery of transcranial electrical stimulation 130 to ongoing oscillations may promote deeper sleep. In particular, the transcranial electrical stimulation 130 may contribute to transitioning from stage 2 to stage 3 sleep and/or to staying in deeper sleep for a longer duration. Graph 900 reflects the results of an experiment testing the closed-loop deep sleep enhancement methodology of the present disclosure on a discrete number of people sleeping through a sleep cycle. As shown in FIG. 13, a control group and a stimulus group was used. Differences in NREM2 and NREM3 are significant between the stimulus and control groups. Sleep stages is indicated on the x-axis 902. The time, incremented in minutes, is shown on the y-axis 904. Time for the experiment participants is denoted as mean plus or minus standard error of the mean (SEM). TST indicates the total time spent in a sleep cycle, the wake state is a state of arousal, NREM stage 1 is a light state of sleep, NREM stage 2 is an intermediate stage of sleep, and NREM stage 3 is a deep state of sleep.

As shown in FIG. 13, the amount of time spent in each sleep stage increases based on application of targeted electrical stimulation 130. In particular, more time is spent in deep sleep NREM stages 3 rather than NREM stage 1 and 2 when the targeted electrical stimulation 130 is delivered. The time spent in deep state NREM stage 3 increased significantly, reflecting the improvement in deep sleep quality and efficacy. As described above, this may result from targeting ongoing slow oscillations occurring in NREM stage 2 or 3 with electrical stimulation 130. In four stage NREM classifications, the deep sleep enhancement of the present disclosure might result in even more robust enhancement in stage 4; that is, time spent in deep sleep NREM stage 4 may experience a greater increase than in stage 3. Additionally, less time was spent in TST overall, suggesting an improvement in the efficacy of deep sleep by using the closed-loop deep sleep enhancement methodology.

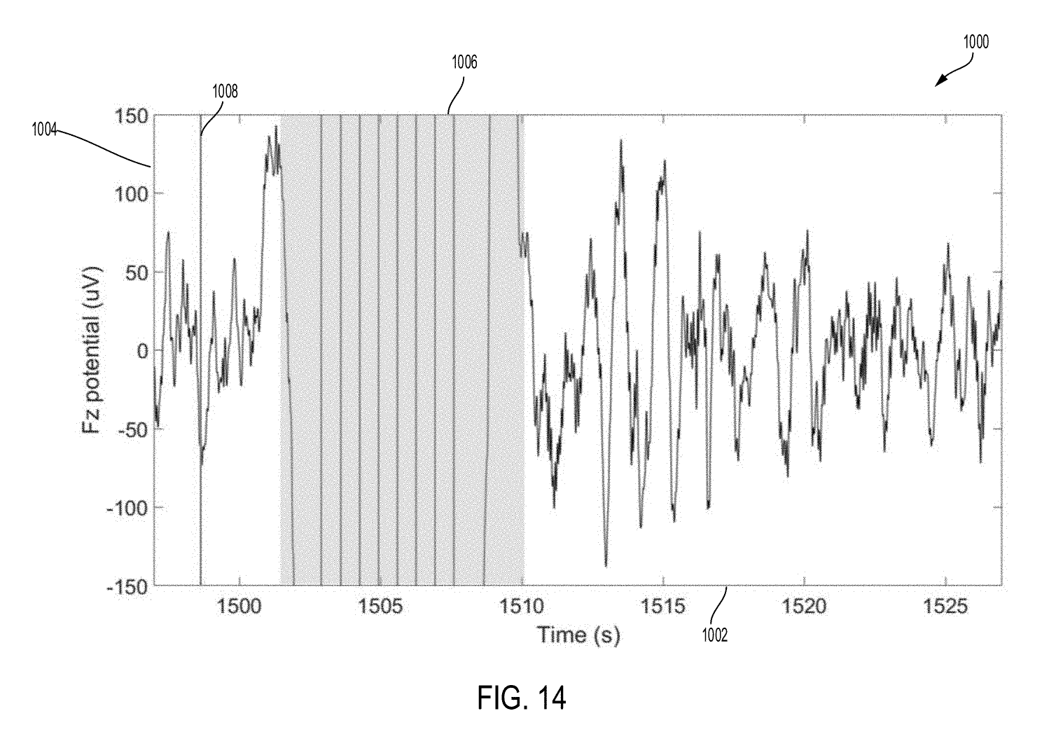

As discussed above, the DUPTs 128 are an alternative to using detected periods of quiescent and/or asynchronous periods of brain activity as the trigger signal for delivering the tES 130. Delivering tES 130 based on detecting DUPTs 128 to increase the power of slow oscillations as well as phase-locked spindle activity during the cortical up-state to enhance deep sleep in this alternative methodology is described above. In contrast to the DUPTs based method, the quiescent and/or asynchronous period based method may increase the rate and duration of slow oscillations occurring subsequent to the tES 130 delivered to the subject. The the computer processing circuit 212 may execute machine executable instructions to deliver tES 130 based on this low spectral power and/or coherence triggers, as described below. These power and coherence measures may be compared by the computer processing circuit 212 to thresholds to determine and confirm that the person's brain is experiencing a period of quiescent and/or asynchronous activity. FIG. 14 is a graph 1000 plotting potential difference relative to time and illustrates a period of time comprising a stimulation cycle, according to one aspect of the present disclosure. On the graph 1000, the x-axis 1002 indicates time in seconds while the y-axis 1004 indicates potential measured at the frontal midline electrode Fz in microvolts (μV). In particular, the graph 1000 illustrates a typical thirty second time window containing a stimulation cycle as implemented by the computer processing circuit 212.

The depicted stimulation cycle comprises approximately eight seconds of stimulation artifacts and twenty two seconds of artifact-free data. The eight seconds comprise five seconds of applied tES 130 by the neurostimulation electrodes 210a-210d, which may be delivered as a series of tDCS or other tES pulses, as well as three seconds for the EEG amplifiers to recover. This eight second period 1006 is highlighted on the graph 1000, and spans from time 1502 seconds to 1510 seconds on the x-axis 1002. As illustrated on the graph 1000, the short duration delivery of slow oscillatory tDCS 130 during the eight second period 1006 results in an increase in the occurrence and duration of slow oscillations. Specifically, a large number of slow oscillations occur in the time period subsequent to the eight second period 1006. Moreover, delivering tES 130 as short duration pulses of repetitive electrical simulation beneficially can enable: a chance to more regularly and consistently investigate the brain's acute response to the tES 130 and to reduce the overall dosage of stimulation applied to the subject. The graph 1000 also includes a line 1008 that indicates the time that a DUPT 128 is detected by the computer processing circuit 212.

The use of tES 130 during low power and synchrony brain activity to promote the subsequent occurrence of slow oscillation has been experimentally tested for validation. In experimental testing, participants received training and were instructed to: take a baseline pre-nap test of tasks to test memory (e.g, test about recalling facts that were earlier presented), take a nap of approximately ninety minutes, take a post-nap memory test approximately thirty minutes after the nap, and take another delayed post-nap memory test approximately forty eight hours later. Participants were divided into a sham group and a short duration test (SDR-tES) group. Participants in both groups were given the EEG headset 104, including the neurostimulation electrodes 210a-210d (connected to the neurostimulator and computer processing circuit 212), and experienced a stimulation acclimation procedure consisting of stimulation at 1 milliampere (mA) or half the maximum current density of 2 mA (which may be the current density used for the delivery of the tES 130). However, participants in the sham group/condition did not receive tES 130 from their EEG headset 104; software was not executed by the computer processing circuit 212 so that tES 130 was not triggered during the nap. In contrast, participants in the SDR-tES group/condition received tES 130 during periods of quiescent and/or asynchronous brain activity. The results of this experimental testing are discussed herein and demonstrate the advantages of SDR-tES to (i) increase the amount of NREM stage 3 sleep relative to NREM stage 1 sleep in a sleep cycle; and (ii) improve declarative memory consolidation to enhance memory and learning for participants.

In one aspect, the tES 130 of the exemplary stimulation cycle portrayed in FIG. 14 may be intermittently triggered based on real-time detection of slow oscillations. Additionally or alternatively, the tES 130 may be triggered independently of the real-time detection of DUPTs 128 and slow oscillations by the computer processing circuit 212; instead, it may be triggered by the computer processing circuit 212 determining the spectral power and coherence measures exceed a threshold. Furthermore, it could be desirable to implement a delay prior to delivering the tES 130. For example, a four second delay could be implemented to avoid interfering with ongoing slow oscillations occurring in the subject's brain. In general, the length of the delay can be determined based on consideration of an approximately one and a half to two second refractory period following each slow oscillation and the observation that slow oscillations can occur in sequence. In this vein, an interarrival time between two adjacent stimulation cycles may be implemented. The length of the interarrival time may be some suitable time determined based on the total amount of stimulation delivered to the subject. One suitable minimum time is thirty seconds between adjacent stimulation cycles. It may also be desirable not to have any delays implemented by the computer processing circuit 212. The lack of delays may be beneficial to better monitoring of the effect of the electrical stimulation.

As the tES 130 is delivered to the subject by the neurostimulation electrodes 210a-210d connected to the neurostimulator, the stimulation may follow a particular current, which may be modeled by finite element modeling (FEM). The FEM may be based on processes of segmentation, electrode generation, co-registration, tessellation, and calculation of the current distribution. For the FEM, the conductive value of air is 3×10⁻¹⁵ Siemens/meter (S/m) while the conductive value of the conductive gel between the Ag/AgCl electrode and scalp is four S/m. The FEM may be performed to generate a gross location of likely current deposition in the brain. The results are summarized in Table 1 below.

TABLE 1
Conductivity values (S/m) for the tissue layers in the tessellated
volume for estimation of tES current distribution.

| WM | GM | CSF | Bone | Skin | Electrodes |
|---|---|---|---|---|---|
| 0.126 | 0.276 | 1.650 | 0.010 | 0.465 | 1.400 |

WM = white matter; GM = gray matter; CSF = cerebrospinal fluid.

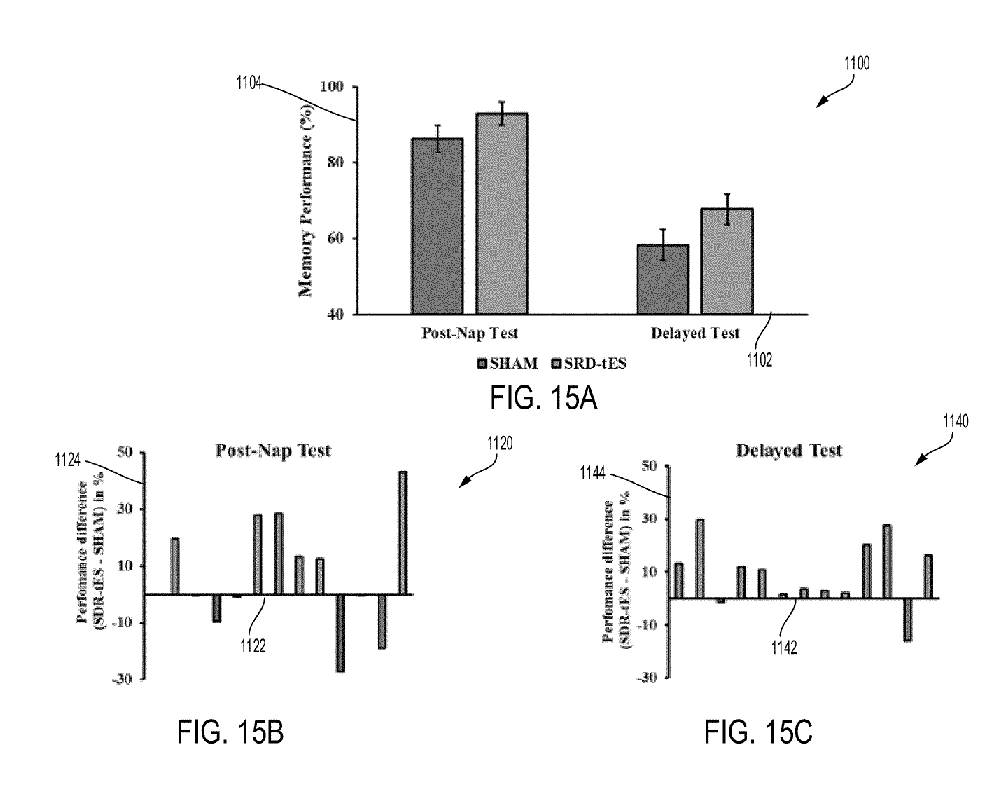

FIGS. 15A-15C are various graphs 1100, 1120, 1140 illustrating differences in memory performance for two groups of subjects at a post-nap and a delayed memory recall test in which the first group of subjects received a transcranial electrical stimulation while the second group of subjects received a sham stimulation, according to various aspects of the present disclosure. The data corresponding to memory participants in sham and SDR-tES conditions in the experimental testing were statistically analyzed using the repeated measures (RM) analysis of variance (ANOVA) technique to determine memory change across sessions and conditions. The results of the RM ANOVA analysis are summarized in Table 2 below.

TABLE 2
Memory performance across tests in the two conditions.

| | |
|---|---|
| Sham | SDR-tES |

Test Performance

| | | |
|---|---|---|
| Immediate Test (%) | 85.4 ± 9.7 | 83.8 ± 11.9 |
| Post Nap Test (%) | 73.8 ± 14.7 | 76.9 ± 11.3 |
| Delayed Test (%) | 50.0 ± 11.7 | 56.9 ± 14.9 |

Performance change

| | | |
|---|---|---|
| Post Nap Performance Change (%) | 82.2 ± 12.6 | 93.0 ± 14.9 |
| Delayed Performance Change (%) | 58.4 ± 11.1 | 67.8 ± 14.4 |

In Table 2, the test performance is computed as the percentage of correctly recalled facts at each testing session. Performance change is computed for each participant as the performance at each test relative to the immediate test, with the immediate test set at 100%. As illustrated by Table 2, no statistically significant difference between the participants in the two conditions/groups is apparent, as determined using ANOVA (Z=0.59, p=0.55). This hypothesis testing result reflects that the immediate memory test was taken prior to the nap so no tES had been delivered to the participants yet. In contrast, both the post-nap test performance and delayed test performance demonstrated statistically significant improvements for the SDR-tES group. This results illustrates the benefits of applying tES (in a closed loop methodology based on spectral power and coherence triggers to cause the computer processing circuit 212 to deliver tES 130 through the neurostimulation electrodes 210a-210d in quiescent and asynchronous periods of neural activity) to improve memory consolidation and recall. In particular, the delayed performance change data in Table 2 represent the improved post-nap and delayed test results for the SDR-tES condition relative to the sham group. That is, participants in SDR-tES group remembered more facts compared to those in the sham group.

The RM ANOVA results are further illustrated in the bar charts of graphs 1100, 1120, 1140. On the x-axes 1102, 1122, 1142, various bars are shown representing the particular memory performance of the sham and SsDR-tES conditions, respectively. The sham condition corresponds to bars of the darker shade while the SDR-tES condition corresponds to bars of the lighter shade. On the y-axes 1104, 1124, 1144, memory performance is indicated as a percentage ranging from 40% to 100% and −30% to 50%, respectively. In FIG. 15A, the bars of the graph 1100 quantify the mean squared error (MSE) of memory at the post-nap and delayed memory test relative to learning performance in the sham and SDR-tES group. Learning performance is set to 100%. As can be readily appreciated, the memory performance is improved for the SDR-tES group; there is a smaller degradation from 100% memory performance for the SDR-tES group compared to the sham group. In FIGS. 15B-15C, the bar of the graph 1120, 1140, show the performance difference for individuals between the sham and SDR-tES conditions at the post-nap test in FIG. 15B and the delayed test in FIG. 15C. A positive percentage indicates that the SDR-tES had better memory performance while a negative percentage indicates that the sham had better memory performance. The graphs 1120, 1140 illustrate that most participants performed better in the SDR-tES condition, which is particularly true for the delayed test.

FIGS. 15A-15C reflect that in the experimental testing, six participants benefitted from SDR-tES while three participants benefitted form the sham at the post-nap test. Moreover, the greater memory retention for SDR-tES was more pronounced at the delayed test, where eleven out of thirteen participants benefitted from the SDR-tES. This is confirmed by the statistical analysis: t₁₃=2.73 and p=0.018 at the delayed test and t₁₃=1.23 and p=0.243 at the post-nap test. As indicated the RM ANOVA, Table 2 and FIGS. 15A-15C, there is a statistically significant decrease in memory performance in the delayed test compared to the post-nap test. Specifically, ANOVA main effect of session indicated F_{1,12}=65.18; p<0.001; and η²ₚ=0.845. The partial eta-squared effect size (η²ₚ) is a measure of effect size. There was also a statistically significant increase in memory retention between sham and SDR-tES condition across the post-nap and delayed test; ANOVA main effect of condition indicated F_{1,12}=5.71; p=0.034; and η²ₚ=0.323. The increased memory retention may result from a greater number of slow oscillation-coupled spindle activations that correspond to the applied SDR-tES 130. These spindle activations can be considered a key mechanism for memory consolidation during sleep. The lack of interaction (F_{1,12}=0.18; p=0.678; and η²ₚ=0.015) may be explained by the greater benefit of the delivery of SDR-tES 130.

Also, the delivery of electrical stimulation 130 results during quiescent and/or asynchronous periods may enhance deep sleep by increasing the amount of time in NREM stage 3 sleep. As discussed in further detail below, these quiescent and/or asynchronous periods of brain activity can be detected by the computer processing circuit 212 monitoring biomarkers to trigger the short durations of repetitive tDCS pulses. Specifically, an empirically determined threshold of measures of spectral power and/or coherence may be used to trigger the SDR-tES 130, such as twice the variance of a root mean square (RMS) power and/or coherence value. The variance could be calculated for a distribution modeling the power and coherence, such as a Gaussian distribution. Thus, when the corresponding thresholds of spectral power and/or coherence are exceeded, the SDR-tES 130 is triggered and applied during the detected low power and synchrony period of brain activity to increase the rate and duration of subsequent slow oscillations. In turn, the relative amount of NREM stage 3 sleep versus NREM stage 1 sleep may increase significantly.

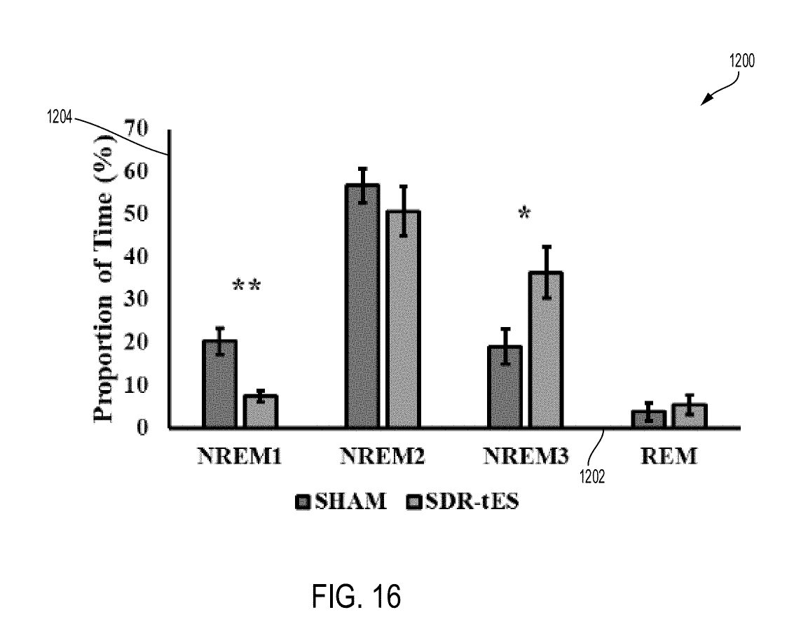

FIG. 16 is a graph 1200 illustrating a comparison between a duration spent in each sleep stage for subjects receiving a transcranial electrical stimulation and subjects receiving a sham stimulation, according to one aspects of the present disclosure. On the x-axis 1202, the various sleep stages are indicated, spanning NREM1, NREM2, NREM3, and REM sleep. On the y-axis 1204, the proportion/percentage of time spent in each sleep stage is indicated by the bars in graph 1200. The first asterisk * corresponds to a p value of less than 0.05 while double asterisk ** corresponds to p<0.005. Each bar in graph 1200 has an associated error bar representing the associated standard error of the mean. The sham condition corresponds to bars of the darker shade while the SDR-tES condition corresponds to bars of the lighter shade. As shown in FIG. 16, it is clear that a greater proportion of the sleep cycle is spent in NREM stage 3 for subjects in the SDR-tES condition. That is, subjects receiving the SDR-tES in periods of quiescent and/or asynchronous brain activity generally spent less than 10% of the sleep cycle in NREM stage 1 but between to 30 to 40% of the sleep cycle in NREM stage 3. Additionally, a smaller portion of the sleep cycle is spent in NREM stage 1 for subjects in the SDR-tES condition. This increase in deep sleep is not shown for subjects in the sham condition. The graph 1200 demonstrates the time spent in NREM stage 1 and NREM stage 3 is similar for sham subjects at approximately generally ˜20% of the sleep cycle each. Thus, FIG. 16 experimentally illustrates the enhancement of deep sleep in the low power and coherence brain activity trigger methodology for delivering tES as implemented by the computer processing circuit 212.

The statistical analysis also illustrates this enhancement of deep sleep, as reflected in Table 3 below.

TABLE 3
Sleep parameters in the two conditions.

| | Sham | SDR-tES |
|---|---|---|
| TST (min) | 80.31 ± 12.40 | 81.00 ± 14.53 |
| SOL (min) | 2.96 ± 4.38 | 2.92 ± 3.92 |
| WASO (min) | 9.38 ± 10.58 | 6.54 ± 6.19 |
| SE (%) | 91.88 ± 7.33 | 86.59 ± 12.64 |
| N1 (%) | 19.67 ± 10.75 | 7.47 ± 4.80 |
| N2 (%) | 56.16 ± 13.16 | 51.27 ± 19.53 |
| N3 (%) | 19.45 ± 13.67 | 36.29 ± 20.27 |
| REM (%) | 4.71 ± 7.57 | 4.97 ± 7.65 |

TST: total sleep time; SOL: sleep onset latency; WASO: wake after sleep onset; SE: sleep efficiency; REM: rapid-eye movement sleep.

As shown in Table 3, the increase in percentage of the sleep cycle in NREM stage 3 increased from 19.45±13.67 for participants in the sham group to 36.29±20.27 for participants in the SDR-tES group. In this connection, the RM ANOVA showed a statistically significant main effect of sleep stage, as indicated by F_{3,13}=31.79; ε<0.51; and ηₚ=0.743 and a statistically significant interaction between sleep stage and condition, as F_{3,13}=6.42; ε<0.61; and η²ₚ=0.369. Epsilon (ε) is a measure of sphericity. Following up on the RM ANOVA test, Fisher's Least Significant Difference (LSD) was used to compare the means of the sleep stage and condition group. The Fisher's LSD post-hoc tests showed a greater proportion of time spent in NREM stage 3 and a lower proportion of time spent in NREM stage 1 for subjects in the SDR-tES condition compared to those in the sham condition, as indicated by values of p=0.017 and p=0.002, respectively.

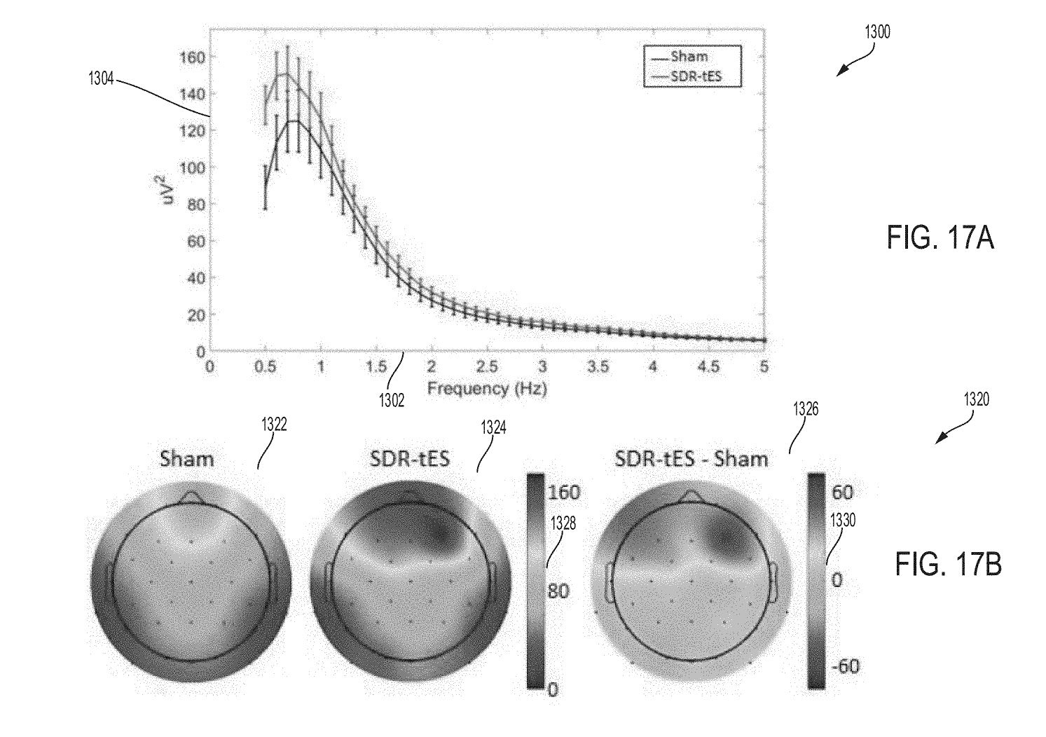

FIGS. 17A-17B are graphs 1300, 1320 of power spectral density and topographic maps of an EEG field in a two dimensional circular view for subjects in sham, electrical stimulation, and the difference between sham and electrical stimulation conditions, according to various aspects of the present disclosure. The graph 1300 depicts the mean power spectral density (PSD) in channel Fz of the EEG headset 104 for both sham and SDR-tES conditions. PSD is plotted for both sham and SDR-tES conditions; and frequency in hertz (Hz) is indicated on the x-axis 1302 while the squared potential in microvolts squared μV² is indicated on the y-axis 1304. PSD is computed over the 0.5-15 Hz range although the x-axis 1302 ranges from 0-5 Hz. PSD may be computed across all sleep stages for sham and SDR-tES conditions to analyze how the delivered SDR-tES 130 affects: the spectral power in the slow oscillation, slow spindle frequency bands (9-12 Hz), and fast spindle frequency bands (12-15 Hz). Slow and fast spindle energy can be measured as z-scores within each corresponding spindle frequency band to reduce variability in signal to noise ratio for smaller amplitude signals across multiple subjects. The graph 1320 depicts the topology of the computed PSD in the one half to one Hz frequency range over the whole head for all subjects in both conditions across all NREM sleep stages. The two sample t-test yielded p=0.37 and therefore no statistically significant difference in the slow oscillation frequency band was discovered between the sham and SDR-tES conditions. Similarly, no statistically significant difference between conditions for the slow or fast spindle bands was discovered.

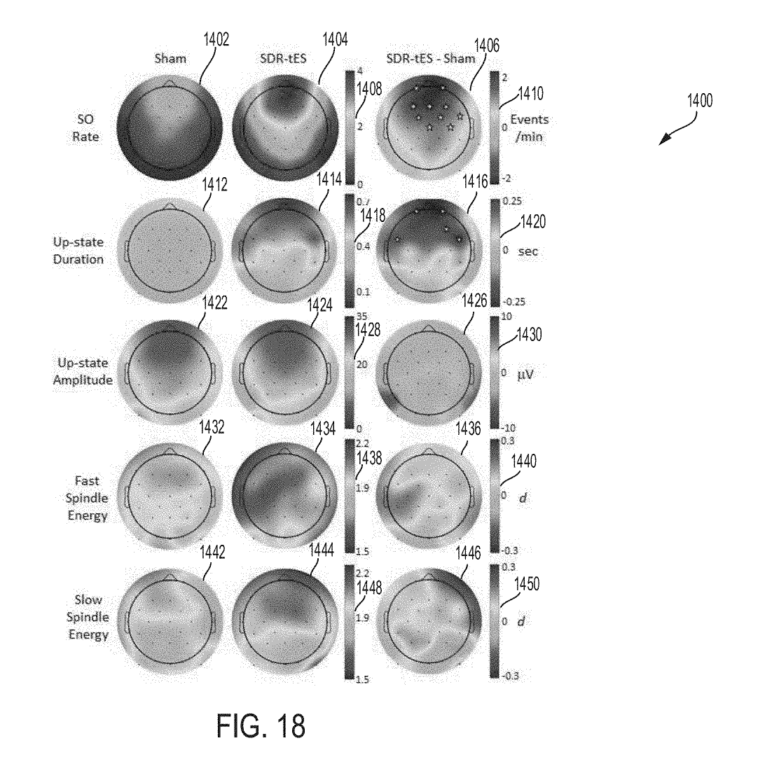

The graph 1320 of FIG. 17B comprises various topoplots 1322, 1324, 1326 showing increases in PSD in the SO frequency band are largest near the anodes 110a-110b and surrounding frontal electrodes. The distribution of PSD values is depicted by the PSD keys 1328, 1330, which range from approximately 0 to 160 and −60 to 60 respectively. The topoplots 1322, 1324, 1326 are plotted topographic maps of EEG fields as two dimensional circular views, which can be generated using MATLAB software (available from MathWords, Natick, Mass.) executed by the computer processing circuit 212, for example. The observed larger PSD in the slow oscillation frequency band corroborates the increases in NREM stage 3 sleep deriving from SDR-tES discussed above. In particular, the graph 1300 illustrates that SDR-tES drives only nominal increases in PSD near the slow oscillation frequency of 1 Hz. The window of the slow oscillation from 0.5-1 Hz is indicated in graph 1300. The graph 1320 more specifically shows average PSD over each electrode (in the 10-20 layout) over 0.5-1 Hz and visually illustrates that all the increases in PSD are over frontal electrodes. Topoplot 1326 visually illustrates the lack of statistically significant differences between sham and SDR-tES conditions. As illustrated by FIGS. 17A-17B, the increase in spectral power occurring at approximately 0.8 Hz may result from the increase in the rate of slow oscillations rather than any change to spectral amplitude. This is illustrated by FIG. 18, as described in more detail below.

FIG. 18 includes topographic maps 1400 of an EEG field in a two dimensional circular view for subjects sham, electrical stimulation, and the difference between sham and electrical stimulation conditions, in which the maps are categorized by slow oscillation rate, up-state duration, up-state amplitude, fast spindle energy, and slow spindle energy, according to one aspect of the present disclosure. The topographic maps 1400 illustrate the effect of delivered SDR-tES 130 on specific biomarkers in sleep associated with sleep-based memory consolidation. Biomarkers for sleep-based memory consolidation may be strongest when nested in slow oscillation cortical up-states. The detection of DUPTs 128 as discussed in connection with FIG. 9 could be used to determine slow oscillation events at each channel of the EEG electrodes. To that end, a −80 μV threshold potential as described in FIG. 9 could be used. However, other suitable threshold potentials such as −50 μV may be used, such as to achieve a better sampling across the EEG electrodes. Any suitable algorithms for automatic labeling of slow oscillation events at each EEG channel may be used. The topographic maps 1400 illustrate analysis of characteristics of up-states and phase-locked sigma activity (in both slow and fast spindle frequency bands) based on these labeled slow oscilations. Specifically, the topographic maps 1400 visually portray the differences between sham and SDR-tES conditions for five different measures: slow oscillation rate, up state duration, up state amplitude, fast spindle energy, and slow spindle energy.

In this way, the topographic maps 1400 illustrate the effect of SDR-tES on DUPT transition-locked oscillatory measures related to memory consolidation during sleep. Topoplots 1402, 1412, 1422, 1432, 1442 show the spatial distribution of these five measures, respectively, for the sham condition. Topoplots 1404, 1414, 1424, 1434, 1444 show the spatial distribution of these five measures, respectively, for the SDR-tES condition. Topoplots 1406, 1416, 1426, 1436, 1446 show the spatial distribution of these five measures, respectively, for the difference between the SDR-tES and the sham condition. The measure keys 1408, 1418, 1428, 1438, 1448 range from 0 to 2 events per minute; 0.1 to 0.7 seconds; 0 to 35 μV; 1.5 to 2.2 d (Cohen's d); and 1.5 to 2.2 d respectively. The measure keys 1410, 1420, 1430, 1440, 1450 range from −2 to 2 events per minute; −0.025 to 0.25 seconds; −10 to 310 μV; −0.3 to 0.3 d; and −0.3 to 0.3 d respectively. Fast and slow spindle energy were measured as z-scores within each spindle's corresponding frequency band, where the differences are indicated as a Cohen's d. The starts in topoplots 1406, 1416 indicates electrode channel locations of statistical significance after adjusting for false discover rate. The p-values for significant electrode locations are listed in Table 4.

| Measure | Location | p-value | Measure | Location | p-value |
|---|---|---|---|---|---|
| Up state | Fp1 | .0077 | SO Rate | Fp1 | .0194 |
| Duration | Fp2 | .0060 | | Fp2 | .0114 |
| | F4 | .0017 | | F3 | .0049 |
| | FC5 | .0048 | | Fz | .0069 |
| | FC6 | .0057 | | F4 | .0014 |
| | | | | FC1 | .0156 |
| | | | | FC2 | .0129 |
| | | | | FC6 | .0159 |
| | | | | Cz | .0257 |
| | | | | C4 | .0098 |

As visually indicated by the topographic maps 1400, application of SDR-tES 130 may significantly increase the rate of slow oscillations by approximately ˜2 slow oscillation events per minute and increase the SO duration by approximately ˜20 ms at frontal electrodes. This demonstrates the advantages of targeted application of SDR-tES 130 during quiescent and/or asynchronous periods of brain activity for enhancing deep sleep and memory recall. The spatial distribution of increases in slow oscillation rate and duration can be strongly observed at locations near stimulation anodes where slow oscillations are normally strongest. SDR-tES also appears to increase DUPT-locked fast and slow spindle energy although these increases were not statistically significant. The nominal increases in fast spindle energy are largest in parietal electrodes, while the increases in slow spindle energy are more globally distributed across channel but are strongest in right frontal electrodes. The effect sizes for all EEG channels are modest (all Cohen's d<0.19), which suggests that SDR-tES may not significantly modulate mean spindle energy. However, despite the lack of increase in mean spindle energy, the increase in overall slow oscillation rates compensates for the lack of increase in energy, and nonetheless may realize a much larger increase in total phase-locked spindle energy.

Furthermore, the four neurophysiological measures except fast spindles in FIG. 18 were averaged across the F₃, F_{z}, and F₄ channels where slow oscillations and slow spindles tend to be strongest. Since fast spindles dominate parietal regions, the average of P3, Pz, and P4 were used for this measure. Of the five measures, only the change in slow oscillation rate is strongly correlated with change in memory recall 48 hours after the initial learning, as described in further detail below. Up-state duration (p=0.31), up-state amplitude (p=0.85), and fast and slow spindle energy measures (p=0.39, p=0.67 respectively) did not show any association with memory performance change. Similarly, there were no significant correlations between any measure and post-nap test performance, in which differences in memory test performance were smaller compared to the delayed test. A positive correlation between differences in slow oscillation rate between the sham and SDR-tES conditions for memory recall in the delayed test may exist; Spearman's Rho correlation might illustrate this correlation. That is, the increase in slow oscillations from the application of SDR-tES might be correlated with greater recall performance.

EEG activity preceding each SDR-tES stimulation cycle was also analyzed to evaluate differences in the timing of SDR-tES 130 application relative to ongoing neural activity. Specifically, the correlation between the spectral power or cross-channel coherence in the one second prior to SDR-tES stimulation 130 and the number of slow oscillation produced in the first 20 seconds after recovery from stimulation artifact in the subset of EEG channels which show the strongest increase in slow oscillation rate (Fp1,Fp2,F3,Fz,F4,FC1,FC2) was analyzed. Spectral power was measured in each EEG channel and averaged across the set. Cross-channel coherence was assessed as the mean of the entire set of pairwise coherence values in the set. A one second window may be selected as the minimum amount of time that would enable assessment of lower frequencies (e.g., 1 Hz) while 20 seconds after the end of the stimulation artifact is almost the minimum duration before a subsequent stimulation cycle. This analysis indicated that the delivery of SDR-tES 130 during periods of asynchronous and/or quiescent brain activity result in increased numbers of subsequent slow oscillations. Based on the negative correlation between the spectral power or cross-channel coherence, the electrical stimulation triggered using the computer processing circuit 212 and delivered through the neurostimulation electrodes 210a-210d may be more effective during these periods of asynchronous and/or quiescent brain activity. To this end, the application of SDR-tES 130 can be triggered upon the computer processing circuit 212 determining that the detected measures of spectral power and/or cross channel coherence have exceed a predetermined threshold. This threshold may be an empirical threshold, such as twice the variance relative to a calculated root mean square (RMS) and fitted probability distribution. This methodology advantageously may improve deep sleep, memory-related sleep physiology, declarative memory consolidation, as well as memory and learning during sleep generally.

Table 5 shows that several spectral bands including delta, beta, and gamma frequency bands were significantly and negatively correlated with the resulting number of slow oscillations occuring immediately following application of SDR-tES 130 in NREM stage 3. Although no significant correlations were found for stimulation in NREM stage 2, SDR-tES 130 may also be delivered in NREM stage 2. For NREM stage 3, both spectral power and mean coherence show this negative correlation, but overall mean coherence features show the strongest correlation with stimulation efficacy. This suggests that stimulating during more quiescent (low power) and less synchronized periods of brain activity lead to larger increases in the slow oscillation rate. Conversely, stimulation during active brain processes and perhaps particularly during an ongoing slow oscillation can reduce the number of resulting slow oscillations. This negative correlation is illustrated by the rand p-values listed in Table 5 below.

TABLE 5
Spectral power and average cross-channel coherence
in low and high frequency bands during NREM stage
3 immediately preceding stimulation is negatively
correlated with the response to stimulation.

| EEG Measure | r | p-value |
|---|---|---|
| Delta Power (1-4 Hz) | −.44 | <<0.01 |
| Delta Coherence | −.38 | <<0.01 |
| Theta Power (5-7 Hz) | 0.1 | 0.08 |
| Theta Coherence | −.03 | 0.41 |
| Alpha Power (8-11 Hz) | −.09 | 0.07 |
| Alpha Coherence | −.05 | 0.28 |
| Sigma Power (12-15 Hz) | −.14 | 0.004 |
| Sigma Coherence | −.18 | 0.06 |
| Beta Power (15-25 Hz) | −.17 | <<0.01 |
| Beta Coherence | −.33 | <<0.01 |
| Gamma Power (25-50 Hz) | −.19 | <<.01 |
| Gamma Coherence | −.38 | <<.01 |
| Total Power (1-50 Hz) | −.43 | <<.01 |
| Total Coherence | −.40 | <<.01 |

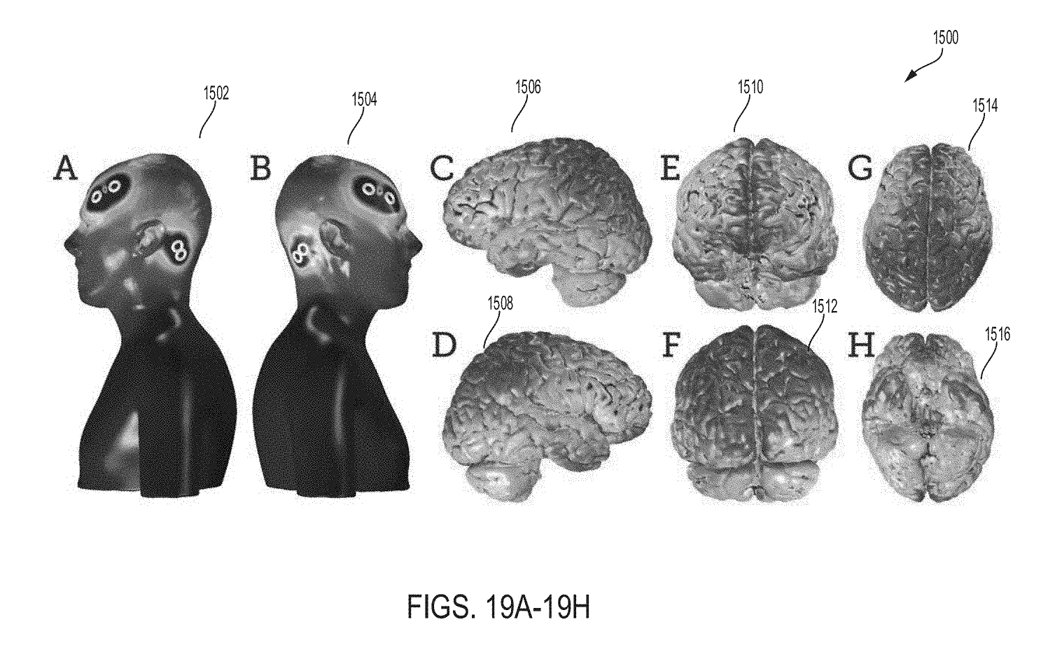

FIGS. 19A-19H are various three dimensional models 1502, 1504, 1506, 1508, 1510, 1512, 1514, 1516 illustrating current distribution on the scalp and gray matter for bilateral and frontal-mastoid electrode locations, according to various aspects of the present disclosure. The three dimensional models 1602-1616 show the current distribution across the cortical surface and ventral brain structures in an exemplary subject. The regions of greatest current distribution appear to be on bilateral frontal cortices approximately under the anodes with weaker activation more posterior towards the cathodes. Strong bilateral frontal activation can be considered consistent with the increases in slow oscillatory power observed over frontal electrodes and provides further evidence that the stimulation may be directly affecting neurophysiology to directly modulate the cortical slow oscillation. The models 1502-1516 also suggest that some of the current may be distributed to the ventral portions of the temporal lobe and the cerebellum.

Accordingly, the delivery of tES 130 through neurostimulation electrodes 210a-210d during NREM stage 3 (or NREM stage 2) while the subject's brain is in a period of low spectral power and/or cross-channel coherence (indicating quiescent and/or asynchronous brain activity, as determined by computer processing circuit 212) may be particularly advantageous for enhancing deep sleep by increasing the proportion of time spent in NREM stage 3 over a sleep cycle. Such delivery may also be particularly advantageous for improving memory consolidation and recall. Both advantages in deep sleep and memory have been experimentally tested and demonstrated, as discussed above. In particular, from a neurophysiological perspective, this low spectral power and/or cross-channel coherence trigger signal methodology for delivering tES 130 may enhance the rate of slow oscillations and the duration of slow oscillation up-states (but not sigma activity). In addition, the increased rate of slow oscillations can also be considered as positively correlated with increased memory performance (particularly at 48 hours after an initial learning task). As previously discussed, the number of slow oscillations immediately following delivery of SDR-tES 130 may be considered negatively correlated with several measures of spectral power and coherence, which suggests the higher effectiveness of applying tES 130 during quiescent and/or less synchronized periods of brain activity.

This low spectral power and/or cross-channel coherence trigger signal methodology may be consistent with a suggestion that when the delivery of stimulation coincides with ongoing slow wave activity, fewer slow oscillations may result. That is, delivery of closed loop tES phase locked to ongoing slow oscillations may reduce the rate of slow oscillations. While the alternative trigger of applying tES 130 based on detecting DUPTs 128 may increase the spectral amplitude of slow oscillations, the low spectral power and/or cross-channel coherence trigger may not effect slow oscillation amplitude. However, delivery of tES 130 during the quiescent and asynchronous periods may cause a spread in the activation of phase-locked slow spindles and fast spindles in frontal, central, and parietal regions respectively, in addition to slow oscillation rate generally. This higher slow oscillation rate very likely may result in a greater number of slow oscillation-coupled spindle activations, which can be considered a key mechanism for memory consolidation during sleep. It is also worth noting that the lack of increase in spectral amplitude of slow oscillations may derive from a delay period (e.g., 4 seconds) in the delivery of tES 130 after occurrence of an endogenous slow oscillation. In general, the delivery of SDR-tES 130 during quiescent and/or asynchronous periods may increase the number of opportunities for slow oscillations for memory consolidation (and more time in NREM stage 3 deep sleep), rather than increasing the efficiency of these existing opportunities. Accordingly, the increase in slow oscillation rate deriving from the delivery of SDR-tES 130 may also correspond to an increase in spectral power across the entire duration of the nap, but this increase would be caused by more slow oscillations rather than the slow oscillations having a higher amplitude. It is also noted that although the tES 130 delivered during quiescent and/or asynchronous periods of brain activity could follow detection of a DUPT 128, the DUPT 128 detection step is not necessary for the low spectral power and/or cross-channel coherence triggered tES 130.

Furthermore, the use of short bursts of slow oscillatory tES (e.g. tDCS) can also be beneficial. Shorter durations or pulses of electrical stimulation triggered by underlying sleep physiology can be more beneficial to the subject receiving the stimulation. That is, the presently disclosed closed-loop methodologies advantageously can result in a large reduction in the total dose of stimulation during sleep. Advantages include lesser irritation to the subject and greater ability to adjust the stimulation. Also, the experimental testing was applied to daytime naps, in which the participants did not spend a significant time overall in NREM stage 3 or overall sleep generally. Indeed, benefits by improving memory-related sleep physiology and declarative memory consolidation of facts could be realized after just a single daytime nap period and reduced electrical stimulation relative to open-loop stimulation approaches. The significant neurophysiological effects of the tES including increased slow oscillation rate and duration may mean that this tES methodology is effective even with a low stimulation dose. Specifically, a required stimulation dosage for effective treatment may be less than a conventional standard of twenty to sixty minutes as the maximum duration of stimulation to incur minimal risk. Elimination of the stimulation artifact may enable a more direct comparison of the results of this low spectral power and/or cross-channel coherence based stimulation to other interventions. The closed-loop deep sleep enhancement methodology described herein may be particularly applicable to older adults who experience reduced deep sleep, which may particularly be the case during daytime sleep. Moreover, the memory testing used in the experimental testing was a declaration task comprising learning a series of facts about destinations around the world, which is different from other standardized lab tasks such as word-pair association tasks and object location tasks. Because the declaration memory task may be considered more similar to a standard educational task, this tES methodology may be effective for improving memory in more standard education tasks. Accordingly, the present disclosure discloses multiple closed-loop deep sleep enhancement methodologies, including a low spectral power and/or cross-channel coherence trigger signal and an alternative DUPT 128 trigger implemented by computer processing circuit 212 for delivering tES 130 through neurostimulation electrodes 210a-210d to the brain.

Turning now to FIG. 12, FIG. 12 illustrates an architectural or component view of a computing system that may be employed with a closed-loop deep sleep enhancement methodology described above according to various aspects of the present disclosure. For example, the computer processing circuit 212 described in the present disclosure may be part of such a computing system 800. In various aspects, as illustrated, the computing system 800 comprises one or more processors 802 (e.g., microprocessor, microcontroller, digital signal processor, logic circuits) coupled to various sensors 804 (e.g., EEG sensors, electrodes) and intervention signal generator 814 (e.g., neurostimulation electrodes, audio, visual, and the like) via a suitable driver 812 circuit. In addition, to the processor(s) 802, a storage device 806 (having operating logic 808) and communication interface 810, are coupled to each other as shown.

A computer processing circuit 800, shown in FIG. 12, comprises one or more microprocessor(s), digital signal processor, memory, input/output (I/O), analog-to-digital converter (ADC) circuits, digital-to-analog converter (DAC) circuits, and other features required of a functional computer to process EEG signals, execute the sleep stage detection algorithm 116, 600, 700, the deep sleep enhancement algorithm 118, and deliver the intervention signal 130, among other functions. The computer processing circuit 800 receives inputs from the EEG sensors 208a-b, processes these signals and converts them into digital waveforms that may be employed by the sleep stage detection algorithm 116, 600, 700, and the deep sleep enhancement algorithm 118.

As described above, the EEG sensors 174 may be configured to detect and collect EEG signals from various locations of the head of the subject 102. As shown in FIGS. 1-7, the processor 802 processes the EEG signals data received from the EEG sensor(s) 804 to convert the signals with an ADC and execute the sleep stage detection algorithm 116, 600, 700 to determine the sleep stage of the subject 102. When the sleep stage is stage 2 or 3, the processor 802 executes the deep sleep enhancement algorithm 118 on the same data to determine whether a large negative potential transition has occurred. The intervention signal generator 814 applies an intervention signal 130 to the subject 102 via the driver 170 circuit. As discussed above, the neurostimulator 814 may deliver an electrical stimulation such as a mild pulsed tDCS stimulation of 2 mA. Accordingly, the computing system 800 may comprise a DAC to generate suitable intervention signals 130.

The processor 802 may be configured to execute the operating logic 808. The processor 802 may be any one of a number of single or multi-core processors known in the art. The storage 806 may comprise volatile and non-volatile storage media configured to store persistent and temporal (working) copies of the operating logic 808.

Additionally or alternatively, the computer processing circuit 800 may deliver the intervention signal 130 according to other triggers besides DUPTs. These other triggers can include biomarkers indicating low brain activity and/or low brain synchronization states. That is, the intervention signal 130 may be gated based on biomarkers implicating cortical down-states. To obtain such biomarkers, in one aspect, the EEG signals and/or digital EEG waveforms 300 can be assessed or otherwise analyzed to generate a measure of total activity of the subject. IN particular, the computer processing circuit 800 may compare measures of spectral power and cross-channel coherence across EEG channels to trigger delivering the intervention signal 130 (e.g., tES 130 through neurostimulation electrodes 210a-210d)

In various aspects, the operating logic 808 may be configured to process the collected EEG signals, such as the EEG signals 302a-s shown in FIG. 3, as described above. In various aspects, the operating logic 808 may be configured to perform the initial processing, and transmit the data, using wired or wireless media, to a separate host computer hosting the application to determine and generate instructions on the process described in connection with FIGS. 1-11. For these aspects, the operating logic 808 may be further configured to receive EEG signals from the subject and provide the processed EEG signals to a hosting computer. In alternate aspects, the operating logic 808 may be configured to assume a larger role in receiving the EEG signals. In either case, whether determined on its own or responsive to instructions from a hosting computer, the operating logic 808 may be further configured to control the neurostimulator 814 to deliver transcranial electrical stimulation to the subject when a transition event has been determined during sleep stages 2 or 3.

In various aspects, the operating logic 808 may be implemented in instructions supported by the instruction set architecture (ISA) of the processor 802, or in higher level languages and compiled into the supported ISA. The operating logic 808 may comprise one or more logic units or modules. The operating logic 808 may be implemented in an object oriented manner. The operating logic 808 may be configured to be executed in a multi-tasking and/or multi-thread manner. In other aspects, the operating logic 808 may be implemented in hardware such as a gate array.

In various aspects, the communication interface 810 may be configured to facilitate communication between a peripheral device and the computing system 800. The communication may include transmission of the collected EEG signals associated with the subject as described herein to a hosting computer, and transmission of data associated with the EEG signals from the host computer to the peripheral device. In various aspects, the communication interface 810 may be a wired or a wireless communication interface. An example of a wired communication interface may include, but is not limited to, a Universal Serial Bus (USB) interface. An example of a wireless communication interface may include, but is not limited to, a Bluetooth interface, Wi-Fi, or the like.

For various aspects, the processor 802 may be packaged together with the operating logic 808. In various aspects, the processor 802 may be packaged together with the operating logic 166 to form a System in Package (SiP). In various aspects, the processor 802 may be integrated on the same die with the operating logic 808. In various aspects, the processor 802 may be packaged together with the operating logic 808 to form a System on Chip (SoC).

Having shown and described various aspects of the present disclosure, further adaptations of the methods and systems described herein may be accomplished by appropriate modifications by one of ordinary skill in the art without departing from the scope of the present disclosure. Several of such potential modifications have been mentioned, and others will be apparent to those skilled in the art. For instance, the examples, aspects, geometrics, materials, dimensions, ratios, steps, and the like discussed above are illustrative and are not required. Accordingly, the scope of the present disclosure should be considered in terms of the following claims and is understood not to be limited to the details of structure and operation shown and described in the specification and drawings.

While various details have been set forth in the foregoing description, it will be appreciated that the various aspects of the system and method for using sleep enhancement during sleep may be practiced without these specific details. One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Further, while several forms have been illustrated and described, it is not the intention of the applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

For conciseness and clarity of disclosure, selected aspects of the foregoing disclosure have been shown in block diagram form rather than in detail. Some portions of the detailed descriptions provided herein may be presented in terms of instructions that operate on data that is stored in a computer memory. Such descriptions and representations are used by those skilled in the art to describe and convey the substance of their work to others skilled in the art. In general, an algorithm refers to a self-consistent sequence of steps leading to a desired result, where a “step” refers to a manipulation of physical quantities which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities.

Unless specifically stated otherwise as apparent from the foregoing disclosure, it is appreciated that, throughout the foregoing disclosure, discussions using terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one form, several portions of the subject matter described herein may be implemented via an application specific integrated circuits (ASIC), a field programmable gate array (FPGA), a digital signal processor (DSP), or other integrated formats. However, those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative form of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In some instances, one or more elements may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other. It is to be understood that depicted architectures of different components contained within, or connected with, different other components are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated also can be viewed as being “operably connected,” or “operably coupled,” to each other to achieve the desired functionality, and any two components capable of being so associated also can be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In other instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present disclosure have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “one form,” or “a form” means that a particular feature, structure, or characteristic described in connection with the aspect is included in at least one aspect. Thus, appearances of the phrases “in one aspect,” “in an aspect,” “in one form,” or “in an form” in various places throughout the specification are not necessarily all referring to the same aspect. Furthermore, the particular features, structures or characteristics may be combined in any suitable manner in one or more aspects.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

All of the above-mentioned U.S. patents, U.S. patent application publications, U.S. patent applications, foreign patents, foreign patent applications, non-patent publications referred to in this specification and/or listed in any Application Data Sheet, or any other disclosure material are incorporated herein by reference, to the extent not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

Various aspects of the subject matter described herein are set out in the following numbered examples:

Example 1. A system for targeted sleep enhancement, the system comprising: a plurality of spatially separated electroencephalography (EEG) sensors configured to be located on the head of a subject to generate a plurality of EEG signals; a plurality of stimulation electrodes configured to be located on the head of the subject; a computer processing circuit configured to: receive and process the plurality of EEG signals; determine that the subject is in a sleep stage 3 based on a specific EEG signal of the processed plurality of EEG signals; determine a period of at least one of quiescent and asynchronous brain activity of the subject, wherein the period is determined based on the processed plurality of EEG signals; and deliver a transcranial electrical stimulation through the plurality of stimulation electrodes during the period of quiescent brain activity.

Example 2. The system for targeted sleep enhancement of Example 1, wherein the computer processing circuit is further programmed to: generate at least one of a measure of absolute spectral power and a measure of cross-channel coherence across the processed plurality of EEG signals; and determine the existence of the period of quiescent brain activity based on at least one of the measure of absolute spectral power and the measure of cross-channel coherence.

Example 3. The system for targeted sleep enhancement of Example 2, wherein at least one of the measure of absolute spectral power and the measure of cross-channel coherence is generated in a gamma spectral band.

Example 4. The system for targeted sleep enhancement of any one of Examples 2-3, wherein the at least one of the measure of absolute spectral power and the measure of cross-channel coherence comprises a threshold to trigger the delivery of the transcranial electrical stimulation.

Example 5. The system for targeted sleep enhancement of any one of Examples 2-4, wherein the at least one of the measure of absolute spectral power and the measure of cross-channel coherence comprises a threshold to trigger the delivery of the transcranial electrical stimulation.

Example 6. The system for targeted sleep enhancement of any one of Examples 1-5, wherein the transcranial electrical stimulation is a transcranial direct electrical stimulation.

Example 7. The system for targeted sleep enhancement of any one of Examples 1-6, wherein the transcranial electrical stimulation is delivered as a series of pulses of transcranial electrical stimulation.

Example 8. The system for targeted sleep enhancement of any one of Examples 1-7, wherein the transcranial electrical stimulation is delivered to increase an amount of time that the subject is in sleep stage 3.

Example 9. A headband configured to be used in conjunction with a computer processing circuit for targeted sleep enhancement and configured to be worn on the head of a subject, the headband comprising: a plurality of spatially separated electroencephalography (EEG) sensors configured to be located on the head of the subject to generate a plurality of EEG signals; a plurality of stimulation electrodes configured to be located on the head of the subject, wherein the computer processing circuit is programmed to: receive and process the plurality of EEG signals; determine that the subject is in one of a non rapid-eye movement (NREM) sleep stage 2 or NREM sleep stage 3 based on a specific EEG signal of the processed plurality of EEG signals; and deliver a series of pulses of transcranial electrical stimulation through the plurality of stimulation electrodes during a period of quiescent brain activity of the subject.

Example 10. The headband of Example 9, wherein the computer processing circuit is further programmed to: generate at least one of a measure of absolute spectral power and a measure of cross-channel coherence across the processed plurality of EEG signals; and determine an existence of the quiescent brain activity of the subject based on at least one of the measure of absolute spectral power and the measure of cross-channel coherence.

Example 11. The headband of Example 10, wherein the at least one of the measure of absolute spectral power and the measure of cross-channel coherence comprises a threshold to trigger the delivery of the transcranial electrical stimulation.

Example 12. The headband of any one of Examples 9-11, wherein the computer processing circuit is further programmed to determine that there is an ongoing slow oscillation based on detection of a cortical down-state to up-state transition event.

Example 13. The headband of any one of Examples 9-12, wherein the plurality of stimulation electrodes comprise a plurality of EEG electrodes, a plurality of electrocardiogram (ECG) electrodes, and a plurality of electrooculogram (EOG) electrodes.

Example 14. The headband of Example 13, further comprising a plurality of spatially separated EOG sensors to generate a plurality of EOG signals and a plurality of spatially separated ECG sensors to generate a plurality of ECG signals, wherein the computer processing circuit is further programmed to perform automated sleep scoring based on the plurality of EEG, ECG, and EOG signals.

Example 15. The headband of any one of Examples 9-14, wherein the plurality of stimulation electrodes comprise four electrodes comprising two anodes and two cathodes; the two anodes are positioned at a Fp1 and a Fp2 EEG channel location on the head; and the two cathodes are positioned ipsilaterally, wherein a first cathode of the two cathodes is positioned at a mastoid location on a same side as a first anode of the two anodes and a second cathode of is positioned at a mastoid location on a same side as a second anode of the two anodes.

Example 16. A method for targeted sleep enhancement using an electroencephalography (EEG) headband comprising a computer processing circuit coupled to a memory storing machine executable instructions, a plurality of spatially separated EEG sensors configured to be located on the head of the subject to generate a plurality of EEG signals, and a plurality of stimulation electrodes configured to be located on the head of the subject, the method comprising: executing, by the computer processing circuit, the machine executable instructions to perform targeted deep sleep enhancement, wherein performing targeted deep sleep enhancement comprises: determining that the subject is in a sleep stage 3 based on a specific EEG signal of the plurality of EEG signals; determining that there is an ongoing slow oscillation based on detection of a cortical down state to up state transition event; and delivering the transcranial electrical stimulation.

Example 17. The method for targeted sleep enhancement of Example 16, wherein the delivery of the transcranial electrical stimulation is targeted to slow oscillations occurring in the head of the subject for increasing the amplitude of the slow oscillations.

Example 18. The method for targeted sleep enhancement of Example 16, wherein the delivery of the transcranial electrical stimulation is targeted during a period of quiescent brain activity of the subject.

Example 19. The method for targeted sleep enhancement of any one of Examples 16-18, wherein the computer processing circuit is further programmed to determine the subject is in the sleep stage 3 based on a weighted delta measure.

Example 20. The method for targeted sleep enhancement of any one of Examples 16-19, wherein the computer processing circuit is further programmed to compute the weighted delta measure based on the following expression:

$${D}_{w} = \frac{D}{G}$$

wherein:

D=weighted delta power over the plurality of frontal EEG signals;

G=gamma power over the plurality of frontal EEG signals and the plurality of occipital EEG signals.

1. A system for targeted sleep enhancement, the system comprising:
a plurality of spatially separated electroencephalography (EEG) sensors configured to be located on the head of a subject to generate a plurality of EEG signals;
a plurality of stimulation electrodes configured to be located on the head of the subject;
a computer processing circuit configured to:
receive and process the plurality of EEG signals;
determine that the subject is in a sleep stage 3 based on a specific EEG signal of the processed plurality of EEG signals;
determine a period of at least one of quiescent and asynchronous brain activity of the subject, wherein the period is determined based on the processed plurality of EEG signals; and
deliver a transcranial electrical stimulation through the plurality of stimulation electrodes during the period of quiescent brain activity.

2. The system for targeted sleep enhancement of claim 1, wherein the computer processing circuit is further programmed to:
generate at least one of a measure of absolute spectral power and a measure of cross-channel coherence across the processed plurality of EEG signals; and
determine the existence of the period of quiescent brain activity based on at least one of the measure of absolute spectral power and the measure of cross-channel coherence.

3. The system for targeted sleep enhancement of claim 2, wherein at least one of the measure of absolute spectral power and the measure of cross-channel coherence is generated in a gamma spectral band.

4. The system for targeted sleep enhancement of claim 2, wherein the measure of absolute spectral power is indicative of total brain activity of the subject and the measure of cross-channel coherence is indicative of total synchronization of brain activity of the subject.

5. The system for targeted sleep enhancement of claim 2, wherein the at least one of the measure of absolute spectral power and the measure of cross-channel coherence comprises a threshold to trigger the delivery of the transcranial electrical stimulation.

6. The system for targeted sleep enhancement of claim 1, wherein the transcranial electrical stimulation is a transcranial direct electrical stimulation.

7. The system for targeted sleep enhancement of claim 1, wherein the transcranial electrical stimulation is delivered as a series of pulses of transcranial electrical stimulation.

8. The system for targeted sleep enhancement of claim 1, wherein the transcranial electrical stimulation is delivered to increase an amount of time that the subject is in sleep stage 3.

9. A headband configured to be used in conjunction with a computer processing circuit for targeted sleep enhancement and configured to be worn on the head of a subject, the headband comprising:
a plurality of spatially separated electroencephalography (EEG) sensors configured to be located on the head of the subject to generate a plurality of EEG signals;
a plurality of stimulation electrodes configured to be located on the head of the subject, wherein the computer processing circuit is programmed to:
receive and process the plurality of EEG signals;
determine that the subject is in one of a non rapid-eye movement (NREM) sleep stage 2 or NREM sleep stage 3 based on a specific EEG signal of the processed plurality of EEG signals; and
deliver a series of pulses of transcranial electrical stimulation through the plurality of stimulation electrodes during a period of quiescent brain activity of the subject.

10. The headband of claim 9, wherein the computer processing circuit is further programmed to:
generate at least one of a measure of absolute spectral power and a measure of cross-channel coherence across the processed plurality of EEG signals; and
determine an existence of the quiescent brain activity of the subject based on at least one of the measure of absolute spectral power and the measure of cross-channel coherence.

11. The headband of claim 10, wherein the at least one of the measure of absolute spectral power and the measure of cross-channel coherence comprises a threshold to trigger the delivery of the transcranial electrical stimulation.

12. The headband of claim 9, wherein the computer processing circuit is further programmed to determine that there is an ongoing slow oscillation based on detection of a cortical down-state to up-state transition event.

13. The headband of claim 9, wherein the plurality of stimulation electrodes comprise a plurality of EEG electrodes, a plurality of electrocardiogram (ECG) electrodes, and a plurality of electrooculogram (EOG) electrodes.

14. The headband of claim 13, further comprising a plurality of spatially separated EOG sensors to generate a plurality of EOG signals and a plurality of spatially separated ECG sensors to generate a plurality of ECG signals, wherein the computer processing circuit is further programmed to perform automated sleep scoring based on the plurality of EEG, ECG, and EOG signals.

15. The headband of claim 9, wherein:
the plurality of stimulation electrodes comprise four electrodes comprising two anodes and two cathodes;
the two anodes are positioned at a Fp1 and a Fp2 EEG channel location on the head; and
the two cathodes are positioned ipsilaterally, wherein a first cathode of the two cathodes is positioned at a mastoid location on a same side as a first anode of the two anodes and a second cathode of is positioned at a mastoid location on a same side as a second anode of the two anodes.

16. A method for targeted sleep enhancement using an electroencephalography (EEG) headband comprising a computer processing circuit coupled to a memory storing machine executable instructions, a plurality of spatially separated EEG sensors configured to be located on the head of the subject to generate a plurality of EEG signals, and a plurality of stimulation electrodes configured to be located on the head of the subject, the method comprising:
executing, by the computer processing circuit, the machine executable instructions to perform targeted deep sleep enhancement, wherein performing targeted deep sleep enhancement comprises:
determining that the subject is in a sleep stage 3 based on a specific EEG signal of the plurality of EEG signals;
determining that there is an ongoing slow oscillation based on detection of a cortical down state to up state transition event; and
delivering the transcranial electrical stimulation.

17. The method for targeted sleep enhancement of claim 16, wherein the delivery of the transcranial electrical stimulation is targeted to slow oscillations occurring in the head of the subject for increasing the amplitude of the slow oscillations.

18. The method for targeted sleep enhancement of claim 16, wherein the delivery of the transcranial electrical stimulation is targeted during a period of quiescent brain activity of the subject.

19. The method for targeted sleep enhancement of claim 16, wherein the computer processing circuit is further programmed to determine the subject is in the sleep stage 3 based on a weighted delta measure.

20. The method for targeted sleep enhancement of claim 19, wherein the computer processing circuit is further programmed to compute the weighted delta measure based on the following expression:
$${D}_{w} = \frac{D}{G}$$
wherein:
D=weighted delta power over the plurality of frontal EEG signals;
G=gamma power over the plurality of frontal EEG signals and the plurality of occipital EEG signals.
